(19) United States
(12) Patent Application Publication
(10) Pub. No.: US 2014/0371690 A1
(43) Pub. Date: Dec. 18, 2014

(54) DISPENSING SYSTEMS

(71) Applicant: Leo Pharma A/S, Ballerup (DK)

(72) Inventors: Peter Sprada, London (GB); Graeme Howard Davies, London (GB); Nicholas Henry Reddall, London (GB); Graham Keith Lacy, London (GB); Steven William McLellan, London (GB); Georgina Rogers, West Sussex (GB); Alun Wilcox, Surrey (GB)

(21) Appl. No.: 14/352,471

(22) PCT Filed: Oct. 18, 2012
(86) PCT No.: PCT/EP2012/070705

§ 371 Date: Apr. 17, 2014

Related U.S. Application Data
(60) Provisional application No. 61/549,952, filed on Oct. 21, 2011.
(60) Provisional application No. 61/587,907, filed on Jan. 18, 2012.
(60) Provisional application No. 61/654,577, filed on Jun. 1, 2012.

(51) Int. Cl. A61M 35/00

(52) U.S. Cl. CPC A61M 35/003
USPC 604/290; 604/310; 604/311

(57) ABSTRACT

A hand-held applicator (10) for self-administration of a semi-solid medicament comprises an applicator head (14) with one or more elongate dispensing nozzles (40) and an applicator body (12) for housing a medicament reservoir, such as a cartridge (20). The applicator has a pump assembly and an actuator (30) for pumping the medicament from the one or more of the dispensing nozzles. The applicator head and applicator body are fixed relative to each other in use such that movement of the nozzle or nozzles away from the scalp or other body area during actuation may be avoided. The applicator may further include an application face for spreading medicament over a body area thereby enabling dual functionality as a scalp and body applicator. A dispensing system in the form of a dispensing head and cartridge therefor is also described and characterised by venting features for improving priming of the dispenser. Such priming means are suitable for use in the applicators of the application.

The present invention relates to dispensing systems in general and more especially to applicators for applying medicated preparations to the scalp and scalp margins, and to other body areas, especially for applying medicated gel to a person suffering from psoriasis. More particularly, the invention relates to dispensing systems and applicators for use with medicament cartridges, and to cartridges for use in the dispensing systems and with the applicators.

Psoriasis occurs when the usual cycle of replacing skin cells is accelerated. While psoriasis presents itself as a skin condition, in recent years it has been established that its origins lie in the immune system. Normally skin cells take about 21-28 days to replace themselves, but in psoriasis sufferers the skin cells can be replaced as rapidly as every 2-6 days. This acceleration results in an accumulation of skin cells on the skin surface, often referred to as psoriasis plaque.

Patches of psoriasis, or plaques, can occur more or less anywhere on the body, but most often occur on the elbows, knees, lower back and scalp. Typically, it appears as patches of raised, red skin covered in silvery white scales. The scales are the accumulation of skin cells that are waiting to be shed and the red patches occur as a result of an increase in the number of blood vessels necessary to support the raised volume of cell production.

Frequently, plaques are itchy and uncomfortable, or worse painful or sore and a range of treatments are available to alleviate the condition. Treatment options include topical therapies, phototherapy, systemic medication and biological injections. Treatment is selected according to the severity of the condition, but in most cases topical therapies are preferred, at least at the outset as they carry lower risks and are more convenient for the patient.

For many psoriasis sufferers, topical therapies, that is preparations applied directly to the skin, are sufficient to keep the condition under control. Conventional preparations include those based on one or more of vitamin D, coal tar, dithranol, vitamin A and steroids and are supplied as semi-solid formulations.

Usually, the formulations are prescribed by medical personnel together with a dosage regime defining the amount of medicament to be applied and the frequency of application. Medications are generally supplied in tubes from which an amount is squeezed out for application to the skin area being treated. Often, the amount to be applied is defined in terms of finger-tip units (FTUs), one finger-tip unit being the distance from the tip of an adult finger to the first crease of that finger, and the number of FTUs to be applied depends on the area of application, so the dose is determined according to the skin area being treated.

A number of psoriasis sufferers interviewed on behalf of the Applicant have indicated that reliable dosing is a significant problem. An FTU is an inaccurate measure since it will vary from person to person. If only a small area of skin is affected, a sub-FTU may be required which again is difficult to measure. Obviously, if insufficient medicament is applied, the affected skin will not be soothed or heal as well as it otherwise might under the correct dosage. On the other hand, application of medicament over and above the prescribed dose can have more serious consequences. For example, overdosing of topical treatments can cause severe irritation and burning. Overdosing of formulations such as steroid creams can result in absorption through the skin and unwanted side effects, such as those that may be experienced when systemic doses of steroids are taken.

One common side effect of regular steroid use is thinning or weakening of the skin. Even if steroid cream is applied to a psoriasis area in the correct FTU dosage, there will often be weakening of the skin at the user's fingertips from using them to rub in the cream. Wearing of gloves to apply steroid medication to avoid this problem is recommended but users find this cumbersome, messy and impractical.

For patients with associated psoriatic arthritis, the action of squeezing a bottle or tube of medication can be awkward and/or painful which adds to the difficulty of dispensing a controlled dose.

Further complications arise when the skin area to be treated includes the scalp or scalp regions, such as on the forehead, neck and behind the ears. Hereinafter, references to the scalp include references to the scalp regions. Self-administration can be problematic because the area being treated may not be readily visible; without assistance, medication may have to be applied “blind” to the back of the scalp. Treating the scalp is further complicated by the need to part the hair to expose the affected area(s), and even then much of the medication inevitably ends up spread over the hair rather than the scalp, thus reducing the effective dosage.

The Applicant has therefore identified a need for a medicament applicator for applying semi-solid preparations, such as psoriasis ointments or gels and the like, to body areas including the scalp which overcomes or at least mitigates one or more of the aforementioned problems.

The term medicaments or preparations used herein is intended to embrace medicated creams, gels, ointments and the like that can flow or be extruded and spread for topical application. The terms are used interchangeably throughout, thus references to ointments, gels or the like should be construed as including other forms of semi-solid preparation. Of course, while the applicators and dispensing systems hereinafter described are particularly intended for use in delivering medicated preparations, they may also be used for delivering non-medicated preparations and such use is embraced in the statements of invention and description of preferred embodiments.

From a first aspect, the invention resides in a hand-held applicator for self-administration of a semi-solid medicament directly to the scalp, the applicator comprising:
an applicator head comprising one or more elongate dispensing nozzles for penetrating the hair and contacting the scalp in the area to be treated;
an applicator body for housing a medicament reservoir;
a pump assembly comprising at least one pump chamber having an inlet for receiving medicament from the medicament reservoir and one or more outlets for discharging medicament to the or each dispensing nozzle on the applicator head; and
an actuator operable on the or each pump chamber for pumping a dose of medicament in the chamber through the or each outlet and dispensing a corresponding medicament dose from the one or more of the dispensing nozzles onto the scalp,
wherein the applicator head and applicator body are fixed relative to each other in use such that movement of the nozzle or nozzles away from the scalp or other body area during actuation may be avoided.

As will be understood, by virtue of the applicator head and body being fixed relative to each other, a user may, for example, first locate a nozzle or nozzles on the scalp in the area to be treated and then deliver a dose of medicament to that area without the nozzle or nozzles moving away from the scalp when the actuator is operated. In this way, the user can be confident that the dose of medicament is delivered to the scalp rather than into the hair. Once the dose has been expelled onto the scalp, the user can then spread the medicament in that vicinity using the nozzle or nozzles. Thus the delivery and application of medicament can be achieved without lifting the nozzles from the scalp.

From another aspect, the invention resides in a hand-held applicator for self-administration of a semi-solid medicament directly to the scalp, the applicator comprising:
an applicator head comprising one or more elongate dispensing nozzles for penetrating the hair and contacting the scalp in the area to be treated;
an applicator body for housing a medicament reservoir;
a pump assembly comprising at least one pump chamber having an inlet for receiving medicament from the medicament reservoir and one or more outlets for discharging medicament to the or each dispensing nozzle on the applicator head; and
an actuator operable on the or each pump chamber for pumping a dose of medicament in the chamber through the or each outlet and dispensing a corresponding medicament dose from the one or more of the dispensing nozzles onto the scalp,
wherein the applicator head is interchangeable on the applicator body to enable the applicator to be modified from a single nozzle applicator to a multi-nozzle applicator and vice versa.

The applicator according to this further aspect can therefore be adapted, either by the user or by the supplier, to suit the severity of the condition being treated. Preferably the applicator head and applicator body are fixed relative to each other in use such that movement of the nozzle or nozzles away from the scalp during actuation may be avoided.

From a yet further aspect, the invention resides in a hand-held applicator for self-administration of a semi-solid medicament directly to the scalp, the applicator comprising:
an applicator head comprising a plurality of elongate dispensing nozzles for penetrating the hair and contacting the scalp in the area to be treated;
an applicator body for housing a medicament reservoir;
a pump assembly comprising at least one pump chamber having an inlet for receiving medicament from the medicament reservoir and one or more outlets for discharging medicament to each dispensing nozzle on the applicator head;
an actuator operable on the or each pump chamber for pumping a predetermined dose of medicament from the chamber through the or each outlet and dispensing a corresponding medicament dose from the one or more of the dispensing nozzles onto the scalp; and
a nozzle selector mechanism adapted to permit medicament to be dispensed from all nozzles when in a first selected mode and to permit medicament to be dispensed from a single nozzle only when in a second selected mode.

The applicator according to this aspect offers ultimate versatility in that a user can select whether to dispense from a single nozzle only, suitable for application in small or awkward areas, or from all nozzles, suitable where the area to be treated is more extensive and more readily accessible, and if desired to dispense a single dose from one nozzle or a multiple of the single dose through all nozzles. Again, the applicator head and applicator body are preferably fixed relative to each other in use such that movement of the nozzle or nozzles away from the scalp during actuation may be avoided.

It will of course be appreciated that a patient may often have patches of psoriasis over a number of body areas, including on the scalp and elsewhere. In the past, treatment has involved the application of one medicated preparation to the scalp and a different preparation to other body areas, for example, the application of a gel formulation to the scalp and a more viscous, creamy formulation to other areas of skin. However, this adds a further level of inconvenience to a user for whom the use of a single formulation irrespective of the area being treated is desirable.

Accordingly, and from yet another aspect, the invention resides in a hand-held applicator for targeted self-administration of a semi-solid medicament directly to the scalp and to other body areas, the applicator comprising:
an applicator head comprising an elongate dispensing nozzle capable of penetrating the hair for contacting the scalp;
an applicator body for housing a medicament reservoir;
a pump assembly comprising a pump chamber having an inlet for receiving medicament from the medicament reservoir and an outlet for discharging medicament to the dispensing nozzle on the applicator head; and
an actuator operable on the pump chamber for pumping a dose of medicament in the chamber through the outlet and dispensing a corresponding medicament dose from the dispensing nozzle onto the scalp or other body area,
wherein the applicator head further comprises an application face for spreading the delivered medicament over a body area to be treated.

The applicator according to this further aspect is particularly suitable for use by patients as both a scalp applicator and body applicator. Specifically, the nozzle is able to penetrate the hair to allow medicament to be delivered directly to the scalp, whereupon the nozzle can also assist in spreading the medicament over the scalp in the same vicinity. Equally, the nozzle is able to deliver medicament directly to another body area, such as on the arms or legs, whereupon the application face can be used for spreading the medicament over the desired body area. Thus the applicator can be used universally by all psoriasis patients, irrespective of whether the plaque region is large or small, on the body or on the scalp. Moreover, where the medicament charged to the reservoir is suitable for use on both the scalp and the body, a patient may use the same applicator to apply the medicament to all affected areas, avoiding the need to have separate applicators for the scalp and the body.

Again, the applicator head and applicator body are preferably fixed relative to each other in use such that movement of the nozzle away from the scalp or skin during actuation may be avoided.

It will be understood that applicators according to all aspects of the invention as described herein may include such an application face, thereby making those applicators also especially suitable for use in applying medicament to body areas other than the scalp.

Various preferred features of the applicators according to any one or all of the above aspects are described below. It will be understood that the features described are generally applicable to all aforementioned aspects of the invention, unless stated to the contrary, and to the dispensing systems that are described more generally hereinafter.

The applicator according to the present invention desirably comprises an airless delivery system whereby the medicament is substantially isolated from contact with air whilst within the applicator. As well as guarding against oxidation and hence deterioration of the medicament through contact with air, providing an airless system also ensures reliability in delivering an accurate dose to the scalp or other body area. Furthermore, with an airless system, the applicator may be held in any orientation whilst dispensing medicament. In other words, the applicator body may be held in a substantially upright orientation, or be inverted, or be in any orientation in between, during application of the medicament, thus helping make application easier for the user.

For helping ensure the reserve of medicament is stored and maintained in an airtight manner prior to use, the medicament is preferably supplied in a cartridge. Thus the applicator preferably includes a cartridge port for receiving a dispensing end of a cartridge charged with medicament, the port being in fluid communication with the or each inlet to a respective pump chamber of the pump assembly. The cartridge port is most conveniently provided on the applicator head. The cartridge port is preferably adapted to engage with the cartridge and seal around the cartridge outlet, for example by means of an interference (push) fit, screw fit or bayonet fit, thereby avoiding leakage of the medicament between the cartridge and cartridge port once the cartridge has been fully fitted. The cartridge port may comprise a seal for sealing with the dispensing end of the cartridge, for example sealing against an external wall of the cartridge, such as around a neck or nozzle portion thereof, or against an internal wall thereof, such as inside the neck portion, or both. In a simple form, the seal may comprise an O-ring seal on the applicator, or, in a preferred arrangement, the cartridge port may include a stopper that seals against an internal wall of the dispensing end of the cartridge, preferably a neck portion. In the latter case, the stopper preferably includes a central bore for permitting medicament to flow from the cartridge to the pump chamber via the inlet. More preferably, the inlet to the pump chamber is integral with the stopper.

Typically, the cartridge will be supplied with a cartridge cap, usually a plastic cap, which is removable prior to fitting the cartridge to the applicator. In order to reduce the risk of damage to the cartridge nozzle or neck in the event that the cartridge is dropped, for example, the cartridge cap is preferably strengthened such as in double walled, most preferably in the form of an H-section. This ensures that if dropped, the outer wall of the H-section absorbs at least some of the energy by means of the plastic deformation rather than the energy being transmitted to the nozzle, and which may otherwise result in nozzle fracture or breakage. Alternatively, instead of a cap or in addition thereto, the cartridge may be provided with a seal, such as a foil seal, over the outlet thereof at its dispensing end which seal is removable prior to fitting or which is piercable by the applicator when the cartridge is fitted thereto.

The cartridge may comprise a collapsible container, such as a bag, typically a foil bag, preferably supplied within a rigid housing (so-called “bag-in-bottle” technology) but more preferably the cartridge comprises a substantially rigid container, such as a tube. Most preferably, the cartridge comprises a tube provided internally with a plunger in sealing contact with the inner tube wall, for example having twin lip seals or other such resilient seal(s), which is drawn up towards the cartridge outlet as medicament is discharged. The plunger maintains an airless environment for the medicament within the cartridge.

In one arrangement the cartridge may be integral with the applicator body, thus the applicator body may consist essentially of the cartridge. Alternatively, the cartridge may be separate from, but accommodated at least partially within, the applicator body. In either case, once a cartridge has been depleted, it may simply be detached and withdrawn from the cartridge port and a fresh cartridge inserted. Thus the applicator head, the pump assembly and where applicable the applicator body may be re-used any number of times. In a further alternative arrangement, the applicator may be fully disposable, the applicator head, pump assembly and actuator being disposed of with the cartridge once the cartridge is depleted. In the latter case, the cartridge may include one-way locking features to prevent re-use.

In a particularly preferred arrangement, the applicator head, pump assembly and actuator are provided as a unit, thereafter also referred to as the head assembly, and the applicator body consists essentially of a medicament cartridge adapted to fit with the applicator head. Thus, the applicator is assembled ready for use by attaching a charged cartridge to the head assembly. More preferably, the head assembly and cartridge are each provided with complementary interlocking or non-return features for preventing separation of the cartridge from the head assembly once the cartridge has been fitted. For example, the dispensing end of the cartridge may be provided with an inwardly directed flange and the cartridge port on the applicator head may be provided with a lip seal, such as on the stopper that is inserted into the neck of the cartridge. As will be understood, once the lip seal has advanced beyond the return flange of the cartridge neck, it is no longer possible to separate the cartridge from the applicator head without destroying the seal or breaking the flange. Alternatively, the stopper may be provided with an annular groove for interlocking with an annular bump on the internal wall of the cartridge neck. Thus once the stopper has been fully inserted into the cartridge neck, the bump is held in the groove and again it is difficult to prise the applicator head off the cartridge without breaking the connection.

In a yet further alternative “once-only” fit arrangement, the cartridge and the head assembly may comprise complementary screw threads whereby the cartridge may be screwed on to the head assembly, and a plurality of spaced apart ribs are provided around the cartridge neck and one or more, preferably two, flexible tabs are provided on the cartridge port whereby the ribs are able to depress and ride over the tabs when the cartridge is screwed on to the head assembly but are unable to depress the tab(s) in the reverse direction, thereby preventing separation (unscrewing) of the cartridge from the head assembly.

On the other hand, where the applicator is intended for re-use and the applicator body is separate from the cartridge, the body may comprise a hollow container, typically a cylinder, open at the distal end (the end furthest from the pump assembly) to allow insertion of the cartridge. Advantageously the cylinder is of a length such that the end of the cartridge, when inserted in the cylinder and fitted to the cartridge port, extends beyond the distal end of the cylinder thus permitting the cartridge still to be gripped whilst it is being secured, for example by push-fitting or screwing, to the cartridge port. For allowing a user to check which medicament is fitted to the applicator, the distal end of the cartridge may carry a visual indication of its contents.

In one arrangement the cartridge has a length that extends beyond the end of the applicator body when fitted such that the exposed length of cartridge is gripped by a user during application of the medicament. Alternatively the cartridge may be substantially enclosed within the applicator body. An end cap, either supplied on the cartridge at the distal end thereof, or as a component of the applicator, may be provided to close the open end of the applicator body with the cartridge retained therein. The end cap may also serve to help retain the cartridge in position with respect to the cartridge port which leads directly or indirectly to the fluid inlet of the pump assembly. The end surface of the end cap for the applicator body or of the non-dispensing end of the cartridge, as appropriate, is preferably flat or otherwise contoured to support the applicator in a substantially upright position, nozzle(s) uppermost, for storage purposes.

As will be appreciated, prior to delivering an initial dose of medicament, the applicator must first be primed so that the pump chamber and dispensing nozzle or nozzles are fully charged with medicament. The priming operation may be automatic or manual, for example manual priming may be performed by operating the actuator to pump the medicament from the applicator body into the pump chamber and out through the or each nozzle; once medicament begins to emerge out of the or each nozzle the applicator is fully primed and ready for use. Any medicament expelled from the nozzle(s) during priming may be wiped away so that the next operation of the actuator will result in a precise known volume, i.e. a dose, of medicament being dispensed. The dose that is dispensed typically comprises medicament from the nozzles, and the volume of the dose dispensed corresponds to the volume of medicament displaced from the or each pump chamber.

Inevitably, due to filling and manufacturing tolerances, the fill height of medicament in the cartridge may vary to such a degree that priming by operation of the actuator alone is impractical. For example, even if only 0.5 cm³ of air is to be evacuated from the cartridge neck itself, this could require approximately 30 pumps of the actuator to displace the air from the cartridge neck and from the pump chamber and nozzle of the head assembly before medicament emerges from the nozzle tip.

Accordingly, as an alternative to priming by operation of the actuator alone, or to reduce the number of times the actuator has to be pumped in a priming operation, the cartridge and applicator head, or at least the cartridge port thereof, may be configured in such a way as to evacuate some or all of the air as the cartridge is fitted to the head. For example, as described hereinabove, the cartridge port may comprise a stopper for fitting into the neck of the cartridge, with the stopper being provided with a vent through which air displaced by the stopper as it is pushed into the neck of the cartridge is forced. displaces air the stopper having a central bore through which medicament from the cartridge is permitted to flow to the pump chamber. The stopper bore may therefore provide the pump inlet to the pump chamber. Preferably, the stopper bore has a first portion of a first diameter and a second portion of a second diameter which is larger than the first diameter, and a bridging portion between the first and second portions forming a valve seat for an inlet valve to the pump chamber. The bridging portion most preferably comprises an inverted, truncated conical surface acting as a valve seat, such as for a spherical or hemispherical valve, especially a ball valve or the like.

The stopper preferably has a length sufficient to displace some or all of the air that may initially be present in the cartridge, typically above the fill level at the dispensing end, as it is advanced into the cartridge neck.

For ease of insertion and to create an effective seal with the cartridge neck, the stopper may be provided with a lip seal around its outer face. The lip seal forms a continuous seal with the cartridge neck yet requires less pressure on insertion than if the entire outer face were itself to provide the seal.

In one arrangement, a side wall of the stopper may be provided with a thread-like channel, such as in the form of a helical lip seal, in fluid communication with the stopper bore for permitting air to escape as the stopper is advanced into the cartridge. Since the medicament is not easily forced through the thread-like channel, when the forward end of the stopper reaches the medicament and starts to displace that, a large back pressure is built up inside the cartridge. This back-pressure may serve to push back the plunger, if provided, at the opposite end of the cartridge, thereby largely avoiding any overspill of medicament during priming. Accordingly, the cartridge preferably has capacity to accommodate such reverse movement of the plunger as may occur during priming of the applicator.

While the stopper may comprise a substantially solid member, it may alternatively comprise a substantially hollow member. For example, the stopper may comprise concentric inner and outer cylindrical walls, joined at the insertion end to prevent ingress of medicament, other than through the central bore created by the inner cylindrical wall that provides the passageway for medicament to be dispensed from the cartridge. The outer cylindrical wall preferably seals against the end surface of the cartridge neck when the cartridge is in its fully inserted position, for example by means of an outwardly directed flange at its end or a lip seal around the outer circumference adjacent the end. Such a hollow stopper may be comprised of a compressible material to enhance the quality of the seal. Irrespective of whether the stopper is of solid or hollow construction, the external or outer wall may narrow in diameter towards the insertion end to provide a taper for facilitating entry into the cartridge neck.

The cartridge port may comprise a cap member that co-operates, such as by interlocking, with the external wall of the cartridge neck to retain the cartridge in sealing engagement with the applicator head. For example, the cap member may have an internal thread that engages with an external thread on the cartridge neck. Such a cap member has an aperture for allowing medicament to flow into the pump chamber.

In one arrangement, the cartridge port may comprise a stopper attached to a cap member such that the stopper is advanced internally and the cap is advanced externally of the cartridge neck. Preferably, when the stopper is provided with an outwardly directed flange, the cartridge is fitted to the cartridge port by initially pushing the applicator head or head assembly onto the cartridge and then screwing the cartridge to secure and tighten the connection, the screwing action also serving to compress the flange between the upper edge face of the cartridge neck and the cap member thereby sealing the cartridge with respect to the head assembly such that medicament may only be discharged via the central bore. As discussed above, any back pressure created during fitting of the cartridge may be released by reverse movement of the cartridge plunger.

In another arrangement, the neck of the cartridge may be double-walled, the walls spaced sufficiently apart to create a chamber therebetween for accommodating any overspill of medicament as the stopper is advanced into the neck of the cartridge and air evacuated. In this way, any medicament that is displaced during priming is held in the chamber between the neck walls and is prevented from leaking from the applicator. In this arrangement, the stopper may further comprise a cap portion, as an alternative to a separate cap member, which provides a seal with the outer neck wall. Further, the cap portion may also create a seal with the inner neck wall so that medicament in the cartridge does not come into contact with any air that remains in the neck chamber, nor can any medicament that has overspilled into the neck chamber be returned to the cartridge. For example, the cap portion preferably has two concentric walls depending from the internal end face thereof, the walls extending so that they seal against the inner and outer neck walls respectively only when the stopper has been advanced almost to its full extent. In this way, the seals are created after the air has been virtually fully evacuated through displacement by the stopper and then by the medicament as the stopper is advanced.

A yet further alternative arrangement for priming the applicator may reside in the configuration of the cartridge cap that seals the cartridge during storage and is removed therefrom prior to the cartridge being fitted to the applicator head. In particular, the cartridge cap may comprise a priming insert for the non-dispensing end of the cartridge, whereby after detaching the cap from the dispensing end it is inserted into the non-dispensing end to advance the cartridge plunger from behind. In this way, the cartridge cap acts as a tool to drive the plunger and thereby evacuate air through the applicator head. The cartridge cap may also be used in this way in conjunction with the other priming arrangements described above. While the cartridge cap/priming insert may be retained in the rear end of the cartridge after priming, it is preferably removed and discarded. In this way, a user of the applicator can view the position of the plunger by looking into the non-dispensing end of the cartridge and thereby gauging the extent to which the cartridge has been depleted.

The priming insert may comprise an outer wall including a first portion having a circumference substantially the same as the outer circumference of the main cartridge body and a second portion having a circumference smaller than the outer circumference to allow insertion into the non-dispensing end of the cartridge body for advancing the plunger. Preferably the second portion has a circumference that permits sliding along the inner cartridge wall, and a length sufficient to advance the plunger to complete priming before the first portion abuts the end of the cartridge whereupon further insertion is prevented. The priming insert preferably includes a threaded portion or the like for engaging with a complementary engaging feature on the cartridge neck and securing the insert to the neck portion as the cartridge cap. Further, the cartridge cap or insert desirably has a recessed portion that extends into the cartridge neck when fitted thereto, the recessed portion occupying a volume of the neck portion thereby to limit the volume of air that resides in the cartridge at the neck portion when the cap is in place. In this way, the medicament within the cartridge is not exposed to a volume of air that may be prejudicial to the quality of the medicament.

Turning to the dispensing nozzle or nozzles of the applicator, these preferably project at an inclined angle with respect to the applicator body and, where more than one nozzle is provided, the nozzles preferably extend parallel with each other. By having the nozzle(s) inclined with respect to the applicator body, a user does not have to reach so far to access awkward areas of the scalp, such as at the rear of the head, the applicator is more comfortable to hold, especially against the scalp, and the nozzle can be gently laid against the skin when used to dispense medicament to a body area. The nozzle tip may project beyond the outer circumference of the applicator body.

In one arrangement, the nozzle or nozzles may project substantially normally from an inclined face of the applicator head. This arrangement is advantageous not only in terms of helping reduce stress, especially when two or more nozzles are being used to massage medicament over the scalp, but also enabling a more direct, unrestricted flow path for the medicament.

As described hereinabove, the applicator may comprise an application face for spreading dispensed medicament over a body area other than the scalp. In such an arrangement, the application face is preferably disposed adjacent the dispensing nozzle(s), preferably adjacent a single nozzle. More preferably, the dispensing nozzle is contiguous with the application face, rather than projecting from a part of the applicator head separate from the application face.

In order to facilitate spreading of medicament, the application face is preferably substantially planar or gently convex. Moreover, to avoid scraping of the user's skin when spreading medicament, the outer edges, or perimeter, of the application face are preferably rounded. Most preferably the application face is substantially circular or ovoid.

The application face may be inclined with respect to the applicator body, such that a user can deliver medicament to the body from the nozzle and spread the medicament using the application face simply by adjusting the orientation of the applicator relative to the body, that is, without requiring relative movement between the applicator and the user's hand. Thus the dispensing of medicament and subsequent spreading thereof on the user's scalp or skin can be achieved without the user needing to alter his grip on the applicator.

In one preferred arrangement, the application face, which typically is planar or gently convex, may include a tapered portion comprising part of the nozzle. In other words, part of the nozzle's external surface comprises an extension of the application face, preferably the application face and nozzle together present a substantially teardrop-shaped face, with the apex comprising the nozzle. When the applicator is stood on end, that is with the non-dispensing end of the cartridge or the end cap of the applicator body acting as a base, the application face and nozzle together preferably comprise an upwardly facing, inclined surface of the applicator.

Other ergonomic arrangements of the applicator face and nozzle are also contemplated, such as the provision of a sideways facing application face, that is an application face lying substantially parallel with the longitudinal axis of the applicator body. In this orientation, the nozzle may still extend substantially continuously with the application face and at an inclined angle with respect to the longitudinal axis. Alternatively, the applicator comprises a pair of opposing sideways facing application faces with the nozzle extending from and substantially continuous with both faces; this arrangement is particularly versatile and easily used by both left and right handed persons.

In a further alternative arrangement, the application face may be provided substantially underneath the nozzle. For example, when stood on its base (such as provided by an end cap on the applicator body or the non-dispensing end of the cartridge), a generally upwardly inclined nozzle overhangs the application face. In such an arrangement, the application face is preferably also upwardly inclined, but in a direction opposite to that of the nozzle.

The bore of the nozzle(s) through which medicament is forced on dispensing may be substantially uniform in cross-section, or have a narrower portion towards the nozzle tip. The bore of the nozzle may in fact be provided by a tubular member or insert within the nozzle. For strength and ease of cleaning, the nozzle may flare outwards at its base (i.e. at the boundary between the nozzle and the applicator/application face), but otherwise have a generally uniform cross-section along a substantial length of its stem. More preferably, however, the external profile of the or each nozzle is tapered such that the nozzle cross-section decreases gradually along its length towards the nozzle tip. Such gradually tapered nozzles have even better mechanical strength, and also provide a smoother, wipe clean surface.

The external nozzle profile may be generally cylindrical, or conical where the nozzle tapers towards its tip, but could equally be flattened in one or more planes, typically when the nozzle extends from and is substantially continuous with the application face. Desirably, as with the application face, the external profile of the nozzle includes only smooth, rounded edges, to avoid causing discomfort to the skin or scalp during application of medicament. Conveniently, the nozzles, or at least a substantial part thereof, may be moulded integrally with the applicator head.

When the nozzles extend from an applicator face, the applicator face is preferably inclined at an angle in the region of 45° to 65°, more preferably 50° to 55° and especially 52°, in relation to the longitudinal axis of the applicator body. Alternatively, when the applicator comprises an application face for spreading medicament, the application face may be substantially parallel with the longitudinal axis of the applicator body or substantially normal to said axis, or any angle therebetween, typically in the region of 20° to 60°. When parallel with the longitudinal axis, the applicator head may be offset with respect to the applicator body.

When more than two nozzles are present, as in an applicator intended primarily for use on the scalp, it is desirable that the tips of all nozzles terminate on the same plane thereby ensuring that all nozzles may simultaneously rest against the scalp. This is important to avoid medicament being dispensed into the hair and for all nozzles to remain against the scalp during the process of spreading the medicament across for desired area. In this regard, three nozzles are especially preferred since they will always be stable on a surface. In such a scalp applicator, where the nozzles project from an applicator face, the Applicant has found that a nozzle angle, that is the angle between the longitudinal axis of the or each nozzle and the longitudinal axis of the applicator body of between about 30° to 40° is suitable, more preferably about 35°.

As will be appreciated, when the nozzles extend from an applicator face, either a single nozzle, or a plurality of nozzles all of equal length, the applicator face will preferably be inclined at the same angle as the nozzle angle. However, if the nozzles are not all of equal length, the applicator face is preferably inclined at a shallower angle than the nozzle angle. For example, the angle between the plane of the applicator face and the plane substantially normal to the longitudinal axis of the applicator body (hereinafter the face angle) may be between about 30° to 60°, more preferably about 45°.

In any event, to ensure effective penetration of the nozzle(s) through most thicknesses of hair, each nozzle preferably has a minimum length of 10 mm, more preferably in excess of 15 mm, and most preferably 18 mm or more. Whilst the length of the nozzle can be increased significantly further, at least in theory, in practice any nozzle that exceeds about 30 mm becomes prone to damage and is not therefore recommended.

When two or more nozzles are present, they are preferably spaced at least 20 mm and less than 40 mm apart. More preferably between about 25 mm and 35 mm apart, especially from 27 mm to 33 mm, the lower end of the range providing a desirable spread pattern (more even scalp coverage) and the upper end of the range providing good handling stability during massaging.

For treatment of the scalp, the nozzle or nozzles will typically be “massaged” against the affected scalp once the medicament has been dispensed therefrom, or be used in a similar fashion to spread the dispensed medicament over a small body area, such as over a small plaque. For this reason, the or each nozzle is preferably provided with a tip of a softer or more resilient material relative to the material used for the major, elongate portion of the nozzle which should be relatively rigid. For example, the nozzle tips may comprise elastomeric pads or the like for permitting medicament to be spread gently over the sensitive area of the scalp. Even when the applicator is used for treatment of other body areas, the provision of a soft, resilient nozzle tip is beneficial for user comfort.

The material selected for the nozzle tip may influence the balance between feel (softness against the scalp or skin) and penetration (parting the hair). For example, a rubber tip may offer better comfort than a polymer tip, but be less efficient at penetration. Moreover, the manner in which the tips are secured to the nozzle stem may also influence the feel of the tips to a user and their penetration ability. Examples of the different ways of securing the nozzle tips to the nozzle stem include use of a headed bolt, an internal rivet and by stretching the tip over the nozzle stem.

To avoid the nozzles being blocked by the scalp or skin on discharge, or becoming blocked by loose plaque such as that generated as the nozzle tip is massaged against the scalp, instead of having an end port/opening, the discharge port may be located transversely thereto. In this way, the medicament may still be discharged directly onto the scalp where the nozzle(s) rests against the scalp, or onto the skin in other body areas, but from one or more side ports. In a preferred arrangement, the or each nozzle comprises a nozzle stem having an end port and a nozzle tip (in the form of an end cap) overlying the end port and having opposing side ports through which medicament exiting the end port is directed.

In order to help avoid ingress of any foreign material into the applicator via the nozzles, the or each nozzle may terminate in an outlet valve, such as a pin valve or a slit valve, that opens under positive pressure exerted on the medicament in the pump chamber to allow discharge of the medicament through the or each nozzle.

It is of course desirable to protect the applicator head and in particular the nozzle(s) when the applicator is not in use, and in this regard the applicator may further comprise a cap or cover for enclosing the applicator head. Preferably the protective cover fits over the applicator head and is retained thereon, for example by complementary engagement features on the cover and applicator head or applicator body, such as by a snap-fit mechanism or screw-cap, or simply by a friction fit. After the applicator has delivered a dose of medicament to the scalp and the nozzles have massaged in the medicament over the desired area, the applicator head, including the nozzles, can be wiped clean and the protective cover replaced.

Advantageously, the cover for the applicator head not only shields the application face but also serves to seal the nozzle tip or tips. Where the applicator head comprises a single nozzle contiguous with the applicator face, the cover may be shaped to encase the nozzle and the application face. More preferably, the applicator cover extends also to shield the actuator lever for avoiding accidental actuation. Such a cover may therefore extend to shield virtually the whole of the applicator head and actuator, or head assembly. By also shielding the actuator lever, inadvertent damage to the actuator when the applicator is not in use may also be avoided. As well as simply shielding the actuator lever to prevent accidental use, the cover may extend to enclose completely the lever.

In a further alternative arrangement, instead of the applicator cover extending to shield the actuator, it may instead include an actuator lock for preventing actuation. For example, the cover may include at least one locking member for insertion behind the actuator, such as under a lever arm thereof, to prevent movement of the actuator. Preferably, the cover actuator lock comprises a pair of arms that may be clipped under opposite sides of the actuator lever, more preferably each arm terminating in a projection, such as a wedge-shaped projection, to substantially inhibit any movement of the actuator lever towards the applicator body or cartridge. Ideally, the arms of the applicator cover lock are sufficiently resilient to permit them to be spread apart during fitting and cause them to spring back behind the actuator lever once released.

The pump assembly of the applicator is preferably enclosed, for example housed within the applicator body or more preferably within the applicator head, particularly as a component of a head assembly, or therebetween, when the applicator is fully assembled for use, thereby ensuring that the components of the pump can be kept clean and hygienic. Both the applicator head, including the nozzle(s) and, where provided, the application face, and the applicator body are preferably contoured in a manner that provides a substantially continuous external surface such that the applicator as a whole may be readily cleaned by wiping.

Advantageously, the or each fluid inlet of the pump assembly has an inlet valve for sealing the inlet from the medicament reservoir, such as from the cartridge, under positive pressure in the pump chamber and opening the fluid inlet under negative pressure in the pump chamber. Thus the or each inlet valve is closed when positive pressure is exerted on the medicament in the pump chamber by the actuator to effect discharge, but is opened when the pump chamber has been discharged and the actuator released. As will be understood, negative pressure created in the pump chamber immediately after discharge of a dose of medicament therefrom and concurrent opening of the inlet valve results in a further dose of medicament being drawn from the medicament reservoir through the inlet valve into the pump chamber so that the chamber and hence the applicator is ready for its next use.

The or each inlet valve preferably comprises a one-way valve, for example a simple flap valve or a check valve, such that medicament advanced to the pump chamber is prevented from being returned upstream of the inlet valve. Thus such a non-return valve ensures that the medicament supply in the reservoir is kept in isolation.

When the applicator includes a cartridge port for connecting with a medicament cartridge, the inlet valve of the pump assembly may be positioned over the fluid outlet of the cartridge port. In a preferred arrangement, the inlet valve is positioned over the central bore of the stopper, more preferably within the stopper bore, and most preferably the central bore includes a shoulder portion against which the valve member, such as the disc or ball, rests or is seated to close the inlet. The valve member may be raised above the shoulder portion under negative pressure within the pump chamber to allow medicament to flow into the chamber. The inlet valve member is preferably spring-loaded permitting opening of the inlet under negative pressure in the pump chamber (and allowing medicament to be discharged into the chamber) against the spring force, and closing the inlet under the action of the spring on equalisation of the pressure. For example, the valve may comprise a valve member, such as a disc, plug, ball or the like, and a separate spring, but may alternatively take the form of a unitary valve moulding comprising both the valve member and spring. Such a unitary valve moulding may include an anchor portion for securing the moulding with respect to the pump chamber and/or cartridge port, a disc or ball portion for closing the inlet, and a spring portion extending between the anchor portion and the disc or ball portion. As will be appreciated, both the anchor portion and spring portion are such as to permit flow of medicament into the pump chamber. In a particularly preferred arrangement, the anchor member of the inlet valve moulding may be captured between the stopper and the cap member of the cartridge port.

The or each pump chamber of the pump assembly into which medicament is drawn and subsequently discharged may comprise a collapsible chamber whereby the volume of the chamber is reduced under a force applied by operation of the actuator. Such a collapsible chamber may, for example, be in the form of a bellows where side walls of the chamber concertina, or in the form of a rolling diaphragm where an end wall of the chamber is advanced or “rolled” inwards with accompanying shortening of the diaphragm side walls, or in the form of a vertical diaphragm which is pressed down by the actuator to reduce the chamber volume. Specifically, operation of the actuator, which may comprise a lever arm and plunger or, in the case of a vertical diaphragm, an L-shaped lever arm (one arm being the lever arm and the other arm acting as the plunger), the lever arm being squeezed towards the applicator body to apply the plunger to the chamber, causes the contraction of the chamber resulting in positive displacement of a predetermined volume of medicament; the volume displaced corresponding to the desired dose of medicament. The displaced volume of medicament is forced out of its chamber through one or more fluid outlets of the pump assembly to one or more dispensing nozzles, and an equivalent volume of medicament is thereby dispensed from the nozzles.

The collapsible chamber, especially one involving a bellows or rolling diaphragm arrangement, may be comprised of a resilient material, preferably an elastomeric material. The chamber is advantageously formed, such as by moulding, in an expanded (not collapsed) configuration, such that after collapsing and release of the actuator, the collapsed chamber wall reverts to its original, expanded shape. In the case of a rolling diaphragm, for example, a negatively wound spring may be included to assist in returning the chamber wall to its original, non-collapsed state and/or to return the actuator to its initial position ready for dispensing the next dose. As the collapsible chamber expands upon release of the actuator, negative pressure is created within the chamber resulting in a further volume of medicament being drawn into the chamber via the fluid inlet ready for delivery of the next dose.

When the collapsible chamber comprises a rolling diaphragm, it is desirable for the chamber to be guided such that it collapses in a substantially linear manner. In this regard, the diaphragm may be substantially surrounded by an outer casing for guiding or restraining the side walls of the chamber to force substantially linear collapsing. Moreover, to permit the actuator to be operable on the collapsible chamber within the casing, the casing is preferably provided with an aperture aligned with a central, longitudinal axis of the diaphragm and a chamber pin is provided that extends through the aperture such that one end of the pin is in contact with the end wall of the diaphragm and the other end is contactable by the actuator. During actuation, the chamber pin is pushed from one end by the actuator and the other end of the pin therefore advances the diaphragm to discharge medicament. Thus, the chamber pin reciprocates along the longitudinal (collapsing) axis of the diaphragm.

The end of the chamber pin that contacts the diaphragm is preferably also retained by the diaphragm, for example by means of complementary engaging features on the pin and diaphragm, so that when the diaphragm springs back to its “uncollapsed” shape after dispensing, the pin remains in position against the diaphragm ready for the next operation. Without being positively retained against the diaphragm, there is a risk that the pin end may lose contact with the diaphragm under the returning force potentially resulting in misalignment with the diaphragm. In a preferred arrangement, the chamber pin includes a groove adjacent the pin end that contacts the diaphragm and the diaphragm includes outwardly directed central recess in its end wall with an internal rib extending into the recess and which mates with the pin groove to retain the pin in position against the diaphragm.

Other forms of pump chamber are also contemplated. For example, as an alternative to one or more collapsible pump chambers, the chamber or chambers may comprise one or more cylinders along each of which a piston is advanced by operation of an actuator lever. Thus, as the piston is advanced along its respective cylinder, medicament is displaced out of the cylinder through an associated fluid outlet. Upon release of the lever, the piston is retracted along its cylinder, such as under the returning force of a spring, and the resulting negative pressure within the cylinder enables the cylinder to be refilled with medicament drawn in via the fluid inlet ready to deliver the next dose. Advantageously, the trailing end of the piston member carries a button, pin or the like which is pressed by the actuator lever to advance the piston. The piston may include one or more lip seals for providing a seal with the cylinder wall to prevent leakage of medicament and ensure the medicament is discharged via the outlet(s) to the nozzle(s).

Preferably the longitudinal axis of the pump cylinder lies transverse to the longitudinal axis of the applicator body (or cartridge), effectively providing a horizontal actuation with respect to the applicator.

The pump assembly may include one or more outlet valves for sealing the fluid outlet from the or each pump chamber to the nozzle(s) under negative pressure within the respective pump chamber and opening the fluid outlet under positive pressure within the respective pump chamber to allow discharge of medicament to the nozzle(s). Having such outlet valve(s) helps avoid any deterioration in the medicament within the pump chamber, substantially preventing medicament that has been discharged to the nozzle(s) from being returned to the pump chamber and keeping the pump chamber airless.

While various forms of valve may be employed, the outlet valve or valves may advantageously comprise a resilient, preferably elastomeric, material, permitting outward deformation of the material away from the or each outlet during application of a discharging force on the pump chamber to enable medicament to pass from the pump chamber(s) to the nozzles. In one arrangement, when the pump chamber is a collapsible chamber, the outlet valve(s) and collapsible chamber may be provided as an integral unit. Indeed, the inlet valve, collapsible pump chamber and outlet valve(s) may comprise a single, integral elastomeric profile, such as one in which the inlet valve is a flap valve formed by punching a flap or the like on a substantially flat portion of the elastomeric profile and the pump chamber and outlet valve(s) are formed on appropriately contoured portions of the profile. In another arrangement, the outlet valve may be the same as or substantially similar to the inlet valve, for example, the outlet valve may also comprise a unitary valve moulding comprising both a valve member and spring as hereinbefore described. With a valve of this type, any anchor portion may be captured between a moulding that comprises part of the pump assembly and another moulding that provides the fluid pathway to the nozzle.

The material of the chamber walls, or a lining thereof, should ideally be one that does not degrade under prolonged contact with the medicament, thereby ensuring that the medicament does not become contaminated with by-products of degradation and also ensuring that the applicator can be re-used over an extended period of time. Preferred elastomeric materials for the collapsible pump option include silicone, fluoroelastomer or thermoplastic elastomer.

The actuator of the applicator according to the invention may be disposed towards the rear of the inclined applicator face, with the pump chamber(s) of the pump assembly interposed between the nozzle(s) and the actuator. When the actuator is located on a side portion of the applicator that in use, i.e. in a dispensing position, faces away from the scalp, as well as the actuator being readily accessible, facing away from the scalp during dispensing and massaging, the actuator is operated by movement substantially in the dispensing direction. When the actuator, pump chamber(s) and dispensing nozzles are substantially aligned in this way, this helps lessen the force required to operate the actuator to dispense medicament. Not only is this is particularly advantageous for those users who suffer, for example, from stiff or painful joints since the effort required for dispensing medication is reduced, it makes the applicator suitable for one-handed operation.

For applicators comprising a single nozzle and an application face substantially continuous with each other, the actuator may be disposed beneath and substantially in line with the nozzle. Where an application face is provided beneath the nozzle, the actuator may be better disposed on the opposite side of the applicator to the application face, to avoid inadvertent operation of the actuator during spreading of medicament by the application face.

The actuator preferably comprises a plunger mechanism for positively displacing medicament from the or each pump chamber. For example, the actuator may comprise a plunger and a button, whereby the button is pressed to advance the plunger and displace medicament from the pump chamber. More preferably, instead of a button, the actuator may comprise plunger and a lever arm, the lever arm providing a mechanical advantage. The lever arm is advantageously inclined from a fulcrum away from the applicator body, such that the plunger acts upon the pump chamber(s) when the lever arm and applicator body are squeezed together. The lever arm may be substantially straight or gently curved, or may be angular, for example L-shaped, with a first leg extending outwards, substantially normal or inclined with respect to the longitudinal axis of the applicator body, and a second leg depending from the first leg (i.e. depending away from the applicator head) substantially parallel with the applicator body. In the latter arrangement, actuation may be performed either by pushing down on the first leg of the lever arm or by squeezing the second leg with the pump body.

Since the size of the area that may require treatment can differ significantly from patient to patient, as can the specific location on the scalp, the applicator may be adapted accordingly. Thus for patients suffering only a mild scalp condition and requiring application of medicament to a relatively small area of scalp, the applicator may comprise an applicator head having a single nozzle. Such a single nozzle applicator head is also useful when the medicament is required to be applied to awkward or hard to reach areas of the scalp, such as behind the ears, or along the hairline. Whilst not being limited thereto, a single nozzle applicator may be used to deliver a dose of medicament in an amount, for example, in the region of 0.05 to 0.1 g per actuation. Smaller or larger doses can be achieved, such as by appropriate adjustments to the volume of the pump chamber.

For patients suffering from a more severe scalp condition, and hence requiring application of medicament over a wider area, the applicator preferably comprises an applicator head having a plurality of spaced apart nozzles, for example with from two to six nozzles, preferably from two to four nozzles, and most preferably three nozzles. The Applicant has found that three nozzles represent a convenient balance for penetrating the hair and spreading the medicament across a wider area, but also three nozzles are preferred due to the stability achieved; three nozzles are always able to be in contact with the scalp. Again, whilst not being limited thereto, a three-nozzle applicator may be used to deliver a dose three times the dose achieved by an equivalent single nozzle applicator, for example in the region of 0.15 to 0.3 g per actuation. As above, adjustments of the pump chamber volume may be utilised to determine the precise dosage.

The face of the applicator head in the case of a multi-nozzle applicator may have a face area roughly similar to the area of scalp to be covered by the medicament dose delivered. For example, a three-nozzle applicator head may have a face area three times larger than the face area of a single-nozzle applicator.

Of course, a patient's condition may change over a period of time such that it may improve from a serious condition to a mild condition, or vice versa, or a patient may have several areas to treat which are appropriate for single and multiple nozzle treatment. In this regard, rather than providing the applicator with a single user specific applicator head, the applicator may comprise a plurality of interchangeable applicator heads each having a different nozzle configuration. Thus, for example, the applicator may be provided with interchangeable single and triple nozzle applicator heads.

Such interchangeable applicator heads may be provided with an integral pump assembly to allow a user more easily to change the applicator from, say, a single nozzle applicator to a multi-nozzle applicator. As will be appreciated, different fluid outlet arrangements may be required according to the nozzle configuration. For example, while the pump assembly for a multi-nozzle applicator may comprise a single outlet from a single pump chamber with fluid passageways branching further upstream of the pump chamber according to the number of nozzles provided, the pump chamber itself may alternatively have a plurality of outlets. Alternatively, more than one pump chamber may be provided, each chamber delivering medicament to a respective dispensing nozzle or group of nozzles under operation of the actuator.

Instead of providing interchangeable heads to cater for different required dosages, the applicator head of the applicator preferably comprises a plurality of dispensing nozzles and a nozzle selector mechanism operable in a first mode to dispense medicament from all the nozzles and in a second mode to dispense medicament from one of the nozzles only. In this way, the user is able to select whether to dispense medicament from a single nozzle, such as when applying to only a small area of scalp, or to all of the nozzles, such as when the area to be treated is more extensive.

It has been found that an applicator head comprising three nozzles creates an effective balance between scalp coverage and hair penetration ability, together with a significant volume of medicament, typically a drop, dispensed per nozzle. Thus, the nozzle selector mechanism in one mode preferably dispenses medicament from a single nozzle and in another mode it dispenses medicament not only from the single nozzle of the other mode, but two additional nozzles. Of course, it will be appreciated that when more than three nozzles in total are provided, the dispensing arrangements adapted accordingly.

Advantageously, the nozzle that dispenses medicament when the selector is set in its second mode, that is the mode for single nozzle dispensing, projects beyond the other nozzles. In other words, the nozzle of the single dispensing nozzle is preferably longer than the other dispensing nozzles. Thus a user is able not only to identify the operative nozzle easily when dispensing from a single nozzle, but is able to direct that nozzle to the scalp area being treated and massage only that nozzle on the scalp. It is therefore possible to avoid the other (inactive) nozzles also being rubbed against the scalp, potentially aggravating the scalp where no problem exists. The single nozzle tip preferably projects by 10 mm to 15 mm beyond the tips of the other nozzles.

In an applicator that can be switched between single and multiple nozzle dispensing modes, the pump assembly may advantageously comprise more than one pump chamber and the nozzle selector mechanism is adapted to discharge medicament from all pump chambers when in the first mode and from a single pump chamber when in the second mode upon operation of the actuator. By having more than one pump chamber and determining whether to discharge medicament from one or all of the chambers, it is possible also to vary the dose of medicament that is dispensed. For example, the dose discharged from all chambers simultaneously will typically be a multiple of the dose discharged from a single pump chamber only. In this way, the nozzle selector mechanism also dictates the dose dispensed.

In one arrangement, the applicator comprises a pump assembly having two pump chambers, one chamber having a capacity adapted for discharging a dose of medicament to a single nozzle and the other chamber having a capacity greater than the capacity of the one chamber for discharging substantially the same dose of medicament to each of two or more further nozzles. Thus, in the case of an applicator with three nozzles, the capacity of the other chamber is substantially twice that of the first chamber.

Conveniently, a nozzle selector mechanism selectively operable on one or all chambers may be integrated with the actuator. For example, the nozzle selector may be integrated with a plunger of the actuator. In a preferred arrangement, the plunger comprises a rotatable element that is selectively operable on one pump chamber or all pump chambers according to the rotation position of the plunger. More preferably, the plunger can also be rotated to a further position in which the plunger is inoperable on all of the pump chambers, effectively providing a locked position of the applicator in which operation of the actuator results in no medicament being dispensed.

When the applicator comprises two (or more than two, though two is the preferred number) pump chambers each in the form of rolling diaphragms with associated chamber pins projecting out from the diaphragm casings, the rotatable plunger may comprise a plunger body, an actuator rod extending from the rear of the plunger body, and three or more apertures in a front face of the plunger body, the apertures spaced in such a way that when rotated to a first plunger position the plunger face advances all chamber pins upon actuation of the actuator rod thereby to discharge medicament from all pump chambers, when rotated to a second plunger position the plunger face advances only the chamber pin associated with the pump chamber that discharges to a single nozzle (the other chamber pin being received in an aperture so that it is not advanced), and when rotated to a third plunger position the plunger face contacts none of the chamber pins (all chamber pins being received in respective apertures) so that no medicament is discharged upon actuation.

A dial is preferably provided to the rear of the plunger body for permitting a user to select the required dispensing mode, the dial having a central aperture through which the plunger or actuator rod extends. The actuator rod preferably has an asymmetric cross-section and the central dial aperture has a corresponding cross-section so that when the dial is rotated to select a particular mode the actuator rod and hence the plunger body are simultaneously rotated.

The dial may also be provided with a tab that extends for a short distance beyond the outer dial circumference for engaging with a complementary recess on the underside of the actuation lever when the dial ring is in the third, non-dispensing (locked) position.

Advantageously, the switching mechanism further comprises an insert interposed between the dial and plunger body, the insert having a plurality of internal projections acting as keys that co-operate with external projections on the plunger body to permit reciprocating (back and forth) movement of the plunger body only when the plunger body is in pumping (single and multiple nozzle dispensing) or locked positions.

Of course, other arrangements for varying the dose dispensed by the applicator may be provided and will now be described. For instance, the applicator may be provided with a separate dose selector mechanism for permitting variation in the amount of medicament dispensed upon operation of the actuator. When the applicator comprises both a nozzle selector mechanism and a dose selector mechanism, the volume of medicament dispensed from all the nozzles may again be a multiple of the volume of medicament dispensed from a single nozzle. Thus the combination of nozzle and dose selector mechanisms offers another means of changing the applicator from a single nozzle discharge to a multiple nozzle discharge, and changing the dose of medicament that is dispensed.

When the dose selector mechanism is separately operable from the nozzle selector mechanism, a user may, for example, initially operate the nozzle selector mechanism to select either single or multiple nozzle discharge, then operate the dose selector mechanism to select the dose to be discharged from the selected nozzle(s).

The dose selector mechanism may operate by altering the degree of actuation of the actuator between first and second operating modes in order to alter the volume of medicament displaced from the pump chamber. For example, when it is desired to dispense medicament from a single nozzle only, the dose selector mechanism may be set to limit the degree of movement of the actuator and hence the volume of medicament displaced from the pump chamber, whereas when medicament is to be dispensed through all nozzles, the dose selector mechanism is set such that movement of the actuator, such as a lever or button thereof, is unrestricted. It will be appreciated that this arrangement may equally be incorporated on applicators that have fixed applicator heads (for example, single nozzle or triple nozzle heads, including heads that incorporate an application face for spreading medicament), and on applicators with interchangeable heads.

The actuator limiting device may take the form of a switch, such as a lever switch or dial switch, which in a first position permits full actuation and in a second position permits only limited actuation. The switch may be carried on the actuator itself or adjacent thereto. For example, when the actuator comprises a lever, the switch may comprise a lever switch carried on the lever itself or on the body of the applicator. In the latter case, the switch may be adapted to move from a first position in which there is no obstruction of the lever to a second position in which the switch partially obstructs movement of the lever towards the applicator body.

The actuator, whether a button or a lever mechanism or another, may include a spring or equivalent, for returning the actuator to a start position ready for the next actuation. If a switch is present to limit the actuator, then the spring will return the actuator to the appropriate position set by the switch.

The applicator may further comprise an actuator lock for guarding against inadvertent dispensing of medicament. Such a lock is usefully engaged when the applicator is packed in a bag, for example for travel, and is especially useful to prevent accidental movement of a lever arm.

The actuator lock may be in the form of a switch which is moveable, such as by sliding or rotation, between a first position in which movement of the actuator, such as a lever arm, is substantially prevented and a second position in which the lever arm is free to pivot. The switch may be located on the applicator body or head, such as on a head assembly comprising the applicator head, pump assembly and actuator, adjacent the free end of the lever arm, for example between the lever arm and body, or on the lever arm. Insignia on the switch, or on the applicator body, head or lever arm adjacent the switch, or any combinations thereof may be provided to indicate the locked and unlocked positions.

In one arrangement, the actuator lock may be integral with the switch that comprises the movement limiting device of the dose selector. In this way, a user can select one of three switch positions, (i) to prevent any movement of the actuator, (ii) to limit the extent of movement of the actuator for dispensing a reduced dose of medicament or (iii) to permit unrestricted movement of the actuator for dispensing the maximum dose of medicament.

The aforementioned actuator locks offer an alternative solution to the rotatable plunger option hereinbefore described, in which the plunger may be rotated to a position where there is no interaction with the or each pump chamber on actuation of the plunger.

In another variation of an applicator that has a plurality of dispensing nozzles and a nozzle selector mechanism for selecting single or multi-nozzle dispensing, the pump assembly may comprise a unitary chamber, such as in the form of a cylinder, having a first fluid outlet at a distal end thereof for discharging medicament to one of the nozzles and a second fluid outlet at an intermediate position along the cylinder for discharging medicament to the remaining nozzles, wherein in a first operating mode the first and second outlet are open to the chamber such that medicament may be discharged from both first and second fluid outlets and in a second operating mode of the second fluid outlet is blocked by the piston in the cylinder such that medicament may be discharged from the first fluid outlet only.

In such an arrangement, the nozzle selector mechanism may involve advancing an actuator lever from a first position in which the piston is fully retracted to allow a complete discharge of medicament in the cylinder through both outlets to a second position in which the piston is partially advanced to a position in which the which the second fluid outlet is blocked by the piston, at which position the cylinder contains a reduced volume of medicament ready for discharging to a single nozzle. Thus the nozzle selection also automatically selects the dose. In switching from multiple nozzle to single nozzle dispensing, an amount of medicament will be dispensed as the piston is advanced to the second position. This “unwanted” medicament can simply be wiped from the nozzle outlets, and at the next and subsequent operation of the actuator, the correct dose is dispensed from one nozzle only.

In another arrangement, the dose selector mechanism comprises a nozzle shut-off (lock-out) valve for preventing fluid flow to all but a single nozzle and a sliding switch on a lever of the actuator mechanism for limiting the degree of permitted movement (rotation) of the lever. In operation, when dispensing is required from a single nozzle only, the lock-out valve is activated and the actuator switch slid up towards the fulcrum to reduce the travel of the lever and hence the distance by which the piston can be advanced.

In a further alternative arrangement, instead of providing a switch on the actuator lever to limit its movement, a switch may be provided on the applicator body or head which can be moved from a first position in which the actuation lever is permitted movement to displace a first, predetermined dose of medicament to a second position in which the switch limits the movement of the actuation lever to displace a second, smaller, predetermined dose of medicament. The switch effectively acts as a wedge between the lever and applicator body or head to control the degree of rotation of the actuator lever. In this arrangement, it is desirable that the applicator further includes a lock-out valve for preventing discharge of medicament to all but one of the dispensing nozzles. Thus, for single nozzle operation, the body switch is slid to its second position and the lock-out valve activated; in this way, a smaller dose may be dispensed from the applicator. Most preferably, the dose dispensed from the single nozzle will be proportionate to the total number of nozzles on the applicator. For a three nozzle applicator, the dose dispensed from the single nozzle will be one-third of that dispensed when all three nozzles are open.

Of course, there are many other arrangements that permit switching between single and multiple nozzle dispensing and indeed preventing dispensing altogether. For example, instead of a single lock-out valve that prevents medicament flow to all but one of the nozzles, an alternative arrangement may comprise multiple lock-out valves operable to close off flow to some or all nozzles. In the latter case, the valves may be provided on an integrated valve switch, whereby movement of the valve switch in one direction sequentially closes each valve. Thus in one extreme position all valves are closed, in the opposite extreme position all valves are open, and in an intermediate position at least one valve is open and another valve is closed. Such an integrated valve switch may be linked to the actuation lever, whereby the position of the lever as determined by the dose selector sets both the dose dispensed (as determined by the extent of movement permissible) and the number of nozzles effectively closed (as determined by the position of the valve switch). Also, as hereinbefore described, the applicator cover for shielding the application face and nozzle when the applicator is not in use may include an actuator lock in the form of an arm or the like that acts as a wedge behind the actuator lever to prevent movement thereof.

In another example, the actuation lever is simply adapted to provide audible or tactile feedback to a user. Thus, as the lever is squeezed towards the applicator body, sequential contact may be made between the lever and a series of projections to produce a sound, usually a “click”, indicating the dose dispensed. Thus at the first “click”, the user will know that a first dose has been dispensed, if the lever is advanced further a second “click” is heard indicating that a dose twice the amount of the first dose has been dispensed, and at the third “click” a dose three times the amount of the first dose has been dispensed, etc. Moreover, if a separate lock out valve is provided, such as one that closes two out of three nozzles, a user may activate the lock out valve and dispense one, two or three doses from a single nozzle corresponding to the number of clicks. Such an arrangement does of course rely on close attention by a user in order to dispense the desired dosage.

In addition to the present invention residing in an applicator for self-administration of medicament as hereinbefore described, the present invention further resides in a method of delivering semi-solid medicament to the scalp or other body area, said method comprising charging the medicament to an applicator body of an applicator, the applicator further comprising an applicator head fixed relative to the applicator body and having one or more elongate dispensing nozzles for penetrating the hair and contacting the scalp, a pump assembly comprising at least one pump chamber having an inlet for receiving a dose of medicament from the applicator body and one or more outlets for discharging medicament to the or each dispensing nozzle and an actuator operable on the or each pump chamber for pumping the dose of medicament in the chamber through the or each outlet, priming the applicator by operating the actuator to transfer medicament from the applicator body to the pump chamber and the or each nozzle, and thereafter delivering medicament by bringing the or each nozzle into contact with the scalp or other body area and operating the actuator to pump a dose of medicament from the chamber through the nozzle onto the scalp or other body area.

As will be appreciated, the charging and priming steps are required only at the outset prior to the first delivery of medicament to the patient's scalp or body area from a new medicament supply. Once charged and primed, the applicator may then be used to deliver further doses of medicament simply by operation of the actuator.

The step of charging medicament to the applicator preferably comprises inserting a dispensing end of a cartridge charged with medicament into a cartridge port on the applicator which cartridge port is in fluid communication with the inlet to the pump chamber.

Additionally, the present invention further resides in an applicator system characterised by an applicator as hereinbefore described having a cartridge port for receiving a dispensing end of a cartridge, the port being in fluid communication with the or each inlet to a respective pump chamber of the pump assembly, and a cartridge charged with medicament.

The present invention also embraces a dispenser system for semi-solid preparations, that is preparations that have properties intermediate solids and liquids, including but not limited to gels, ointments, creams and the like. In particular, the invention may also be expressed in terms of a system for dispensing a semi-solid preparation comprising:
a dispensing head and
a cartridge for the preparation having a dispensing nozzle for engaging with the dispensing head and a plunger for advancing along the cartridge as the preparation is dispensed,
wherein the dispensing head comprises a cartridge port including a stopper for insertion into and sealing against the cartridge nozzle as the cartridge is engaged with the cartridge port, the stopper having a bore therethrough to permit the preparation to flow from the cartridge to the dispensing head via an inlet valve during a dispensing operation, and
wherein the stopper bore is provided with a vent and the dispenser head includes a passage having a constricted section in fluid communication with the vent, whereby preparation displaced by the stopper upon insertion into the cartridge nozzle is forced into the bore and out through the vent into the constricted passage whereupon the pressure required to force further preparation into the passage becomes greater than the pressure required to move the cartridge plunger and any further displaced preparation is accommodated in the cartridge through reverse movement of the plunger.

As will be understood, by means of the aforementioned dispensing system, as the stopper of the dispensing head is inserted into the cartridge nozzle as would occur on assembly of the two components, air in the nozzle above the preparation fill line is purged through the vent and flows freely through the passage, including through the constricted section, prior to displacement of any preparation. Thus the purging of air in this way on assembly of the cartridge and dispensing head provides for efficient dispensing thereafter. Specifically, the need for priming the resulting assembly is substantially avoided or at least significantly reduced. Moreover, it is possible to assemble the system without significant loss of preparation from the cartridge, since the constricted passage effectively ensures that displaced preparation is mostly kept within the cartridge by expansion of the cartridge volume as the plunger is forced back. The presence of the constricted passage means that only a relatively small volume of preparation is capable of being vented from the cartridge.

Cartridges of the type described are typically subjected to automated filling processes but manufacturing tolerances in preparation density, variation in filling line and part size all may affect the resulting fill level of preparation in the cartridge. As a result, the volume of air in the cartridge nozzle above the fill level which is required to be displaced before preparation can flow into the dispensing head may be subject to wide variation. Accordingly, the stopper preferably has a length such that when fully inserted within the cartridge nozzle its leading end projects beyond a minimum fill level of the cartridge. In this way, the stopper ensures that on assembly air above the fill line is substantially displaced together with a volume of preparation. In a particularly preferred arrangement, the stopper extends substantially along the full length of the cartridge nozzle when fully inserted.

In practice, the volume of preparation displaced will depend on the actual fill level, but the capacity of the passage, at least that part of the passage that is before (upstream of) the constricted section is determined to accommodate a relatively small volume of preparation, preferably a volume that is less than the total volume of preparation that would be displaced by the stopper if the cartridge were filled only to the minimum fill level. The capacity of the passage, including the chamber where provided, is preferably more than about 0.5 cm3, that being the typical difference in volume between minimum and maximum fill levels, and hence the approximate maximum volume of air that will be displaced. In a dispenser having a pump chamber for dispensing doses of preparation of approximately 0.05 cm3, such a passage capacity means that at least 10 pumps of the dispenser may be avoided in a priming operation.

Preferably, the inlet valve is provided within the stopper bore, for example, there may be a valve seat projecting into the bore from the inner bore wall against which a valve member rests to close the valve. The valve seat preferably comprises an inclined surface and the valve closing member preferably comprises a ball. The inlet valve is preferably spring loaded, and the spring component which may be integral with or separate from the valve closing member, such as the ball, is preferably also accommodated within the stopper bore.

As will be appreciated, it is important that those components of the dispensing system that come into contact with the preparation are made of a material compatible with the preparation. In this way, contamination of the preparation may be avoided as well as any deterioration of the dispenser parts. This is especially important In relation to the valve, since any incompatibility with the valve closing member or spring could result in failure of the valve and leakage of the preparation from the dispenser. Accordingly, the ball is preferably a metal or plastics ball, for example made of low density polyethylene (LDPE) or fluorocarbon elastomer (FKM rubber), and the spring may also be of metal or plastics, LDPE being especially preferred. Where the ball is spherical and made of moulded plastic, it may be necessary to post-process the ball after moulding to remove any imperfections around the mould lines that might otherwise compromise the integrity of the ball. Alternatively, the ball may be moulded with an integral peg for the spring and in which case, the need to post-process the spherical surface of the ball can be avoided. Further preferred details regarding the valve are provided herein elsewhere, it being appreciated that those details and the details above are equally applicable to the dispenser system and the pump applicator as described in accordance with the various aspects of the invention.

In a preferred arrangement, the vent is located adjacent the inlet valve. By such means, the bore may be substantially fully charged with preparation upstream of the inlet valve as the stopper is advanced into the cartridge nozzle. More preferably, the bore contains an inner tube of restricted diameter and length (that is a diameter and length less than the diameter and length of the bore) extending from the inlet valve. For example, the inner tube may extend into the bore from the valve seat. Most preferably, the inner tube extends into the bore beyond the position of the vent, but terminates substantially short of the end of the stopper, preferably terminating short of the midpoint of stopper. When the dispenser head and cartridge are assembled, the restricted diameter of the inner tube means that preparation displaced by the stopper flows along the bore as far as the inner tube whereupon the preparation is forced around the outside of inner tube to the vent and passage beyond. In other words, the inner tube acts in the manner of a straw.

The length of the inner tube is desirably such as to have only a small volume so that after assembly of the cartridge and dispenser head, the first actuation of the dispenser (such as by pumping) results in any residual air being purged from the inner tube and preparation being sucked into the inner tube up to the inlet valve. In this way, the valve closing member, typically a ball, will be immediately wetted by the preparation giving an improved seal and thereby making priming of the dispenser more efficient.

Preferably the air and preparation is vented to a first passage section permitting free flow of vented air and preparation followed by a second section which is constricted relative to the first section permitting free flow of air but providing resistance to the flow of preparation and thereby creating back pressure when preparation flows into it. The back pressure results in reverse movement of the plunger in the cartridge to accommodate further displaced preparation.

The passage may comprise a continuous space created between the stopper and a dispensing head manifold, which together preferably define the cartridge port, or may comprise one or more channels between the stopper and manifold. The passage of constricted section may, for example, be provided in the form of one or more channels of smaller cross-section than in the first section, more preferably taking the form of a complementary threaded section between the stopper and manifold, effectively creating an elongate helical channel. The continuous change in direction of the passage provided by the helical channel creates the constriction that resists the flow of the preparation and gives rise to back pressure as preparation is forced along the channel.

In a preferred arrangement, the stopper may comprise a double-walled member wherein the inner wall provides the central bore through which preparation passes from the cartridge to the dispenser head, such as via an inner tube in the bore as hereinbefore described, and the outwardly directed surface of the outer wall seals against the inner wall of the cartridge nozzle, for example, by means of a lip seal or the like. The gap between the inner and outer stopper walls creates a substantially U-shaped cylindrical channel, closed at the front end to force preparation into the central bore as the stopper is inserted into the cartridge nozzle. In this arrangement, the vent is provided through the inner wall of the stopper, whereby air and preparation displaced by advancement of the stopper in the cartridge nozzle are initially forced along the bore towards the vent before exiting the vent for the passage.

The passage is most conveniently provided by a gap or space between the stopper and manifold. For example, the channel of a double-walled stopper may be adapted to receive and accommodate a tubular portion of the dispenser head manifold, and the tubular portion and cylindrical channel are dimensioned relative to each other such as to create the passage therebetween through which air and preparation is forced via the vent. For example, the passage follows a path initially down along the outwardly facing surface of the inner stopper wall, along the base of the channel and up along the inwardly facing surface of the outer stopper wall. Advantageously, the constricted section of the passage is created by complementary threaded sections on the inwardly directed face of the outer stopper wall and outwardly directed face of the manifold tubular portion, preferably adjacent the open end of the U-shaped channel.

Advantageously, the dispensing head further includes a chamber into which displaced air may be contained, such a chamber is preferably provided downstream of the constricted passage, such as at the end of the aforementioned threaded section. The chamber is preferably sealed by the cartridge nozzle when the cartridge is assembled with the dispensing head. In this way, the preparation within the dispenser is effectively contained within a sealed system and any loss of efficacy of the preparation due to oxidation is substantially avoided. In a preferred arrangement, the manifold may include a continuous wall depending into the chamber which wall seals against the nozzle when the cartridge is in its fully inserted position. The manifold sealing wall is preferably concentric with the tubular portion that extends into the channel of the double-walled stopper.

The manifold may additionally include connecting features for securing the cartridge to the dispensing head, for example having a threaded portion for screwing the dispenser head on to a complementary threaded portion on the cartridge, preferably around the base of the cartridge nozzle. Preferably the manifold and cartridge are provided with a one-way lock mechanism permitting assembly of the dispenser head and cartridge but preventing their separation once assembled. For example, the manifold may include one or more, preferably an opposing pair, of head locking clips that ride over engagement ribs on the cartridge (such as are provided on an outer ring, concentric with the nozzle), when the cartridge is screwed on to the dispenser head, but act as a barrier to the ribs when an attempt is made to unscrew the assembly.

When the manifold forms part of an applicator head as hereinbefore described, it may incorporate the pump chamber of the pump assembly. Similarly, the manifold may house an outlet valve of the pump assembly.

It will be understood that the dispensing system according to this further aspect of the invention may be incorporated in the applicators as hereinbefore described, but are not limited to use in such applicators. In particular, the dispensing system may be deployed with any manner of dispensing heads, irrespective of whether the preparation is dispensed from a nozzle or from other forms of openings in the dispenser head, and irrespective of whether the dispenser head is adapted to deliver a dosed volume of preparation, such as from a pump chamber, or otherwise. Of course, the dispensing system of the present invention is especially suitable for use in the applicators according the other aspects of the invention and it should be understood that the dispensing system according to this further aspect may comprise some or all of the essential, optional and preferred features of the applicators set forth herein. Equally, the applicators according to the other aspects of the invention may comprise some or all of the essential, optional and preferred features of the dispensing system.

For the avoidance of doubt, the term semi-solid preparation used herein in relation to the dispensing system is intended to embrace both medicated and non-medicated preparations, that can flow or be extruded or spread, including but not limited to gels, creams, ointments and the like.

The invention will now be described by way of example only, with reference to the following drawings in which:

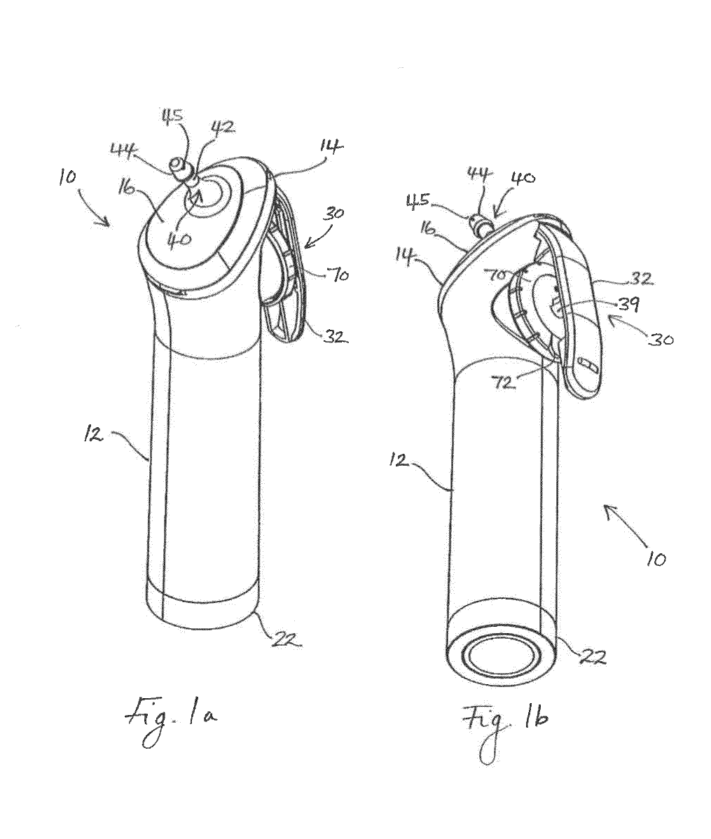

FIGS. 1a to 1d are views of a single nozzle scalp applicator with cartridge in accordance with a first aspect of the invention;

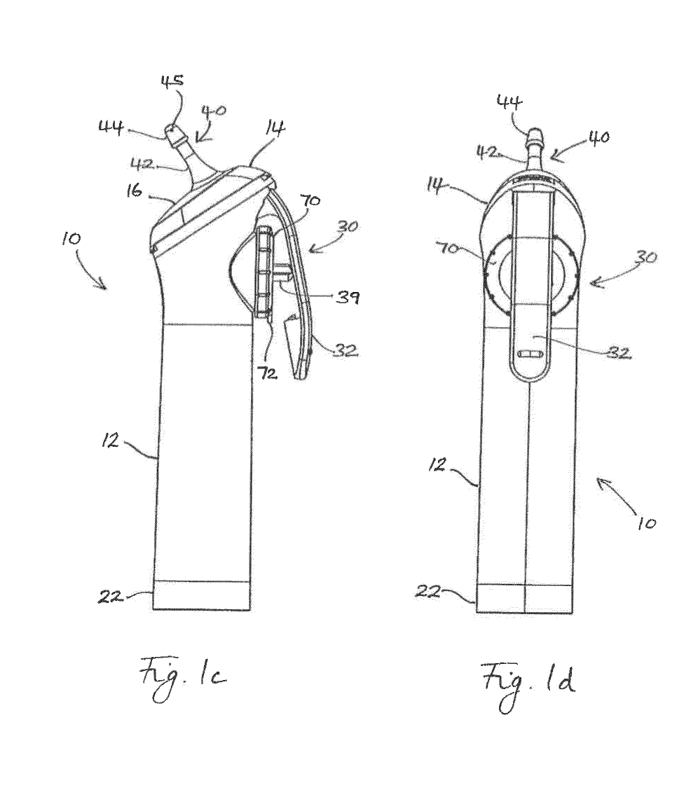

FIG. 2 is a perspective view of the applicator of FIGS. 1a to 1d without the cartridge;

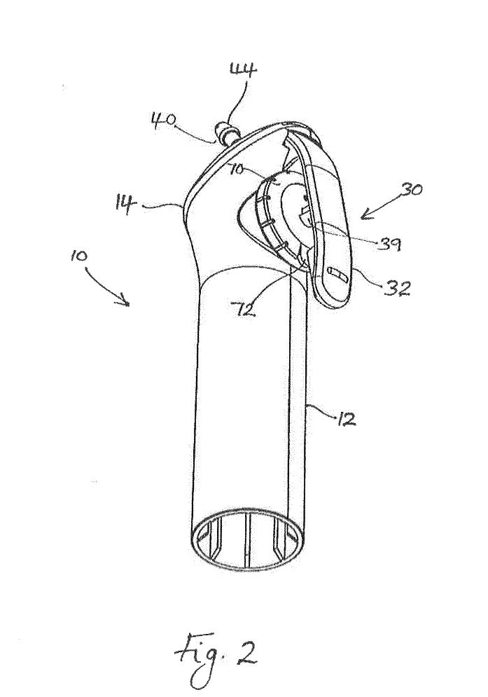

FIGS. 3a to 3e are views of a triple nozzle scalp applicator with cartridge in accordance with another aspect of the invention;

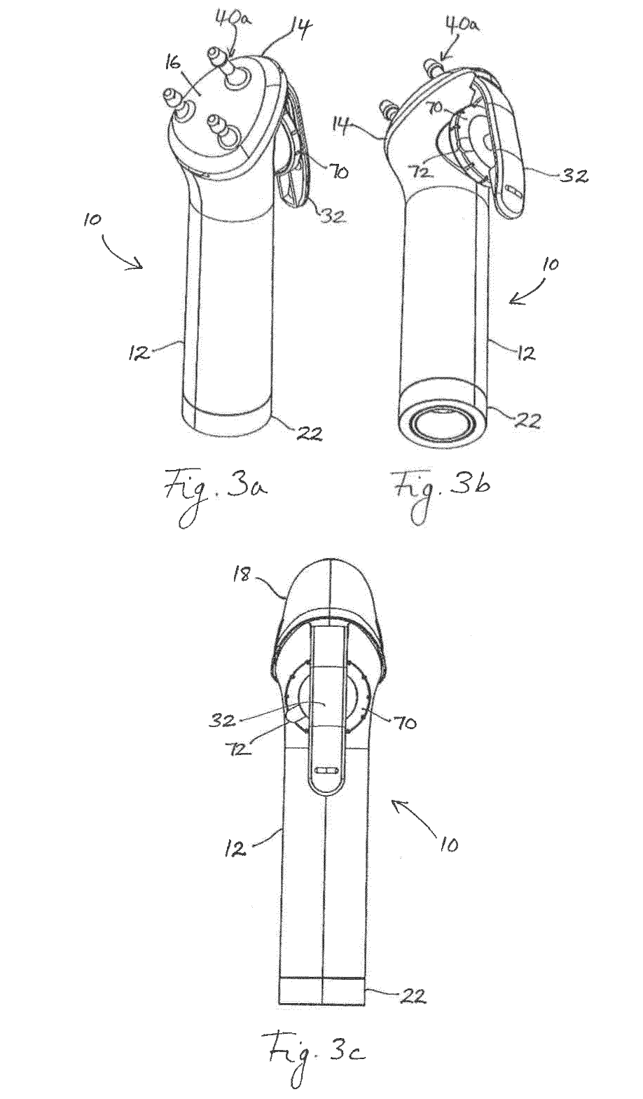

FIG. 4 is a perspective view of the applicator of FIGS. 3a to 3e without the cartridge;

FIGS. 5a to 5c are views of a switchable applicator with cap;

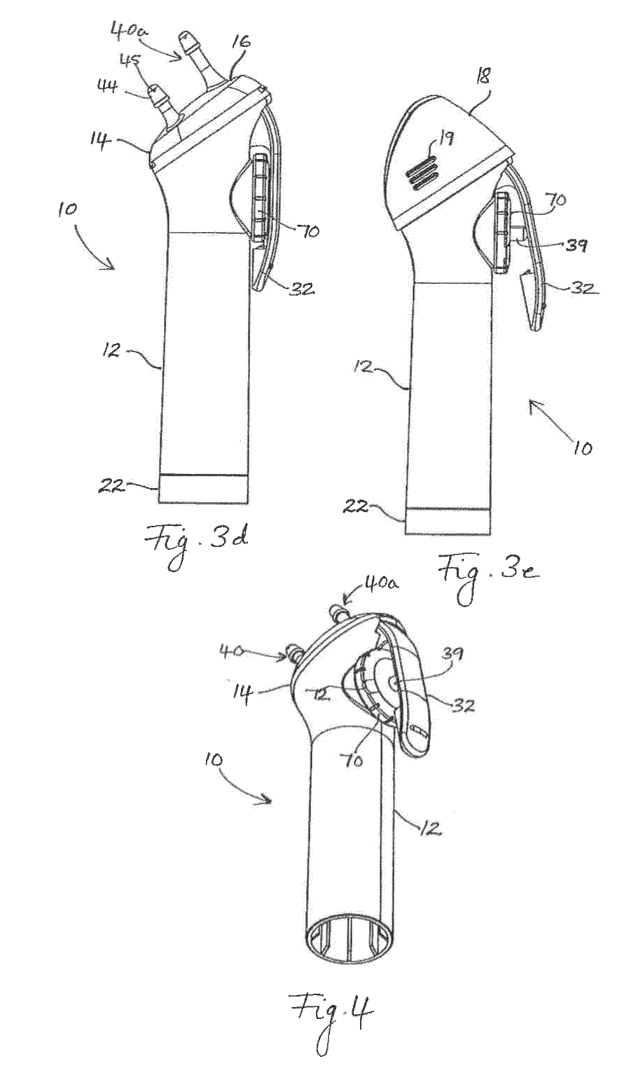

FIGS. 6a to 6c are views of a cartridge with cap for use with the applicators according to the invention;

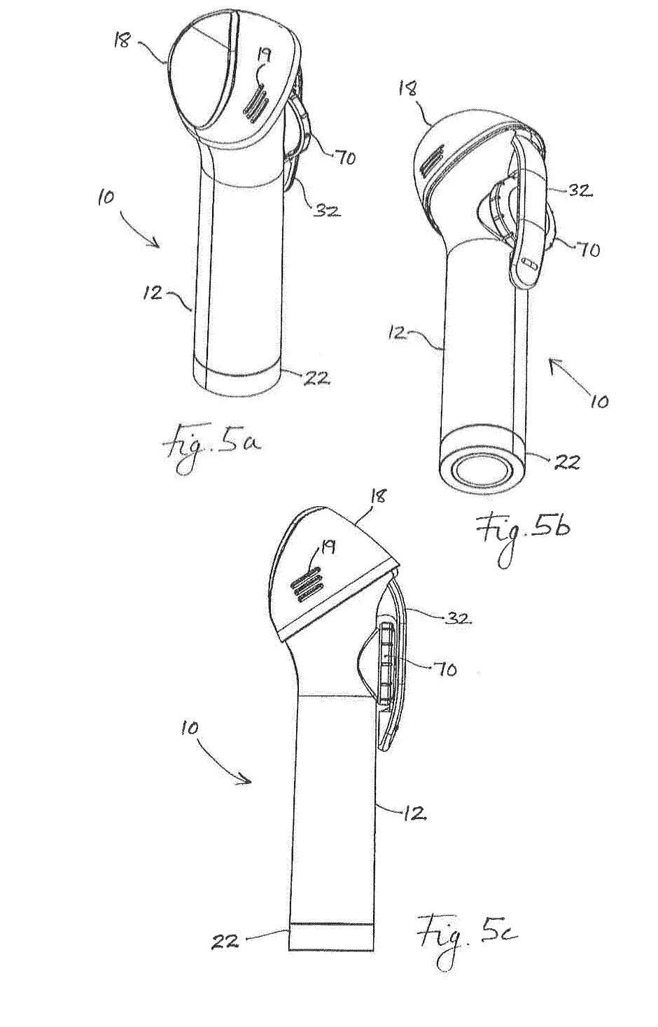

FIGS. 7a to 7c are views of the cartridge of FIGS. 6a to 6c without cap;

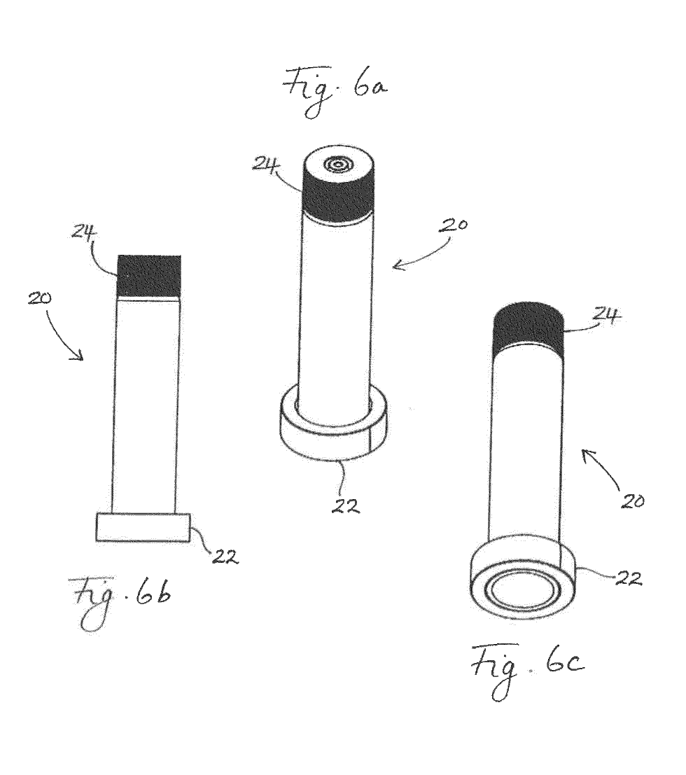

FIG. 8 is a cross-section of the applicator according to FIGS. 3a to 3e fitted with a cartridge but with the pump assembly, plunger and dial omitted;

FIG. 9 is partial cross-section of the applicator according to FIGS. 3a to 3e including the pump assembly, plunger and dial;

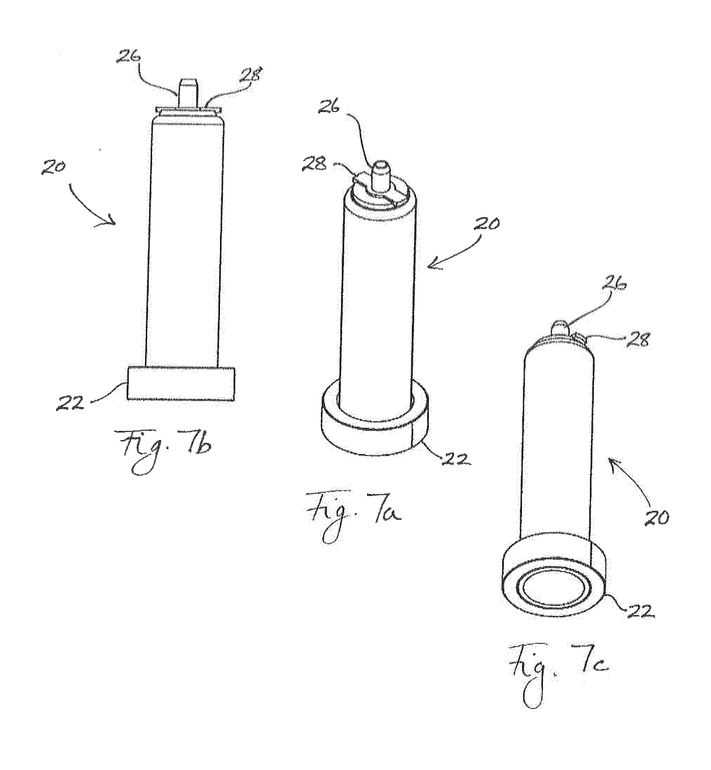

FIG. 10 is an enlarged cross-section of the applicator head of the applicator of FIGS. 3a to 3e;

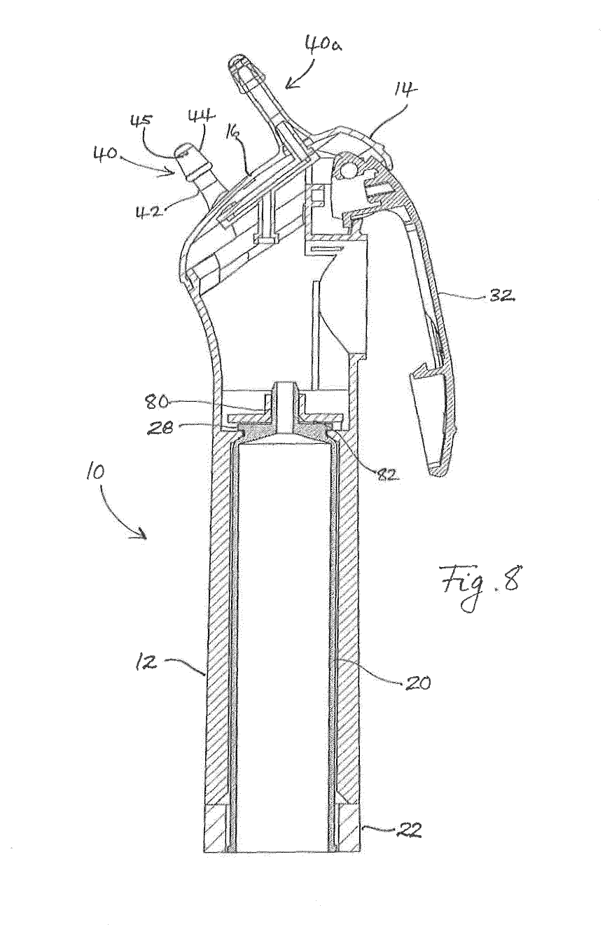

FIG. 11 is an enlarged partial cross-section of the pump assembly of the applicator of FIGS. 3a to 3e;

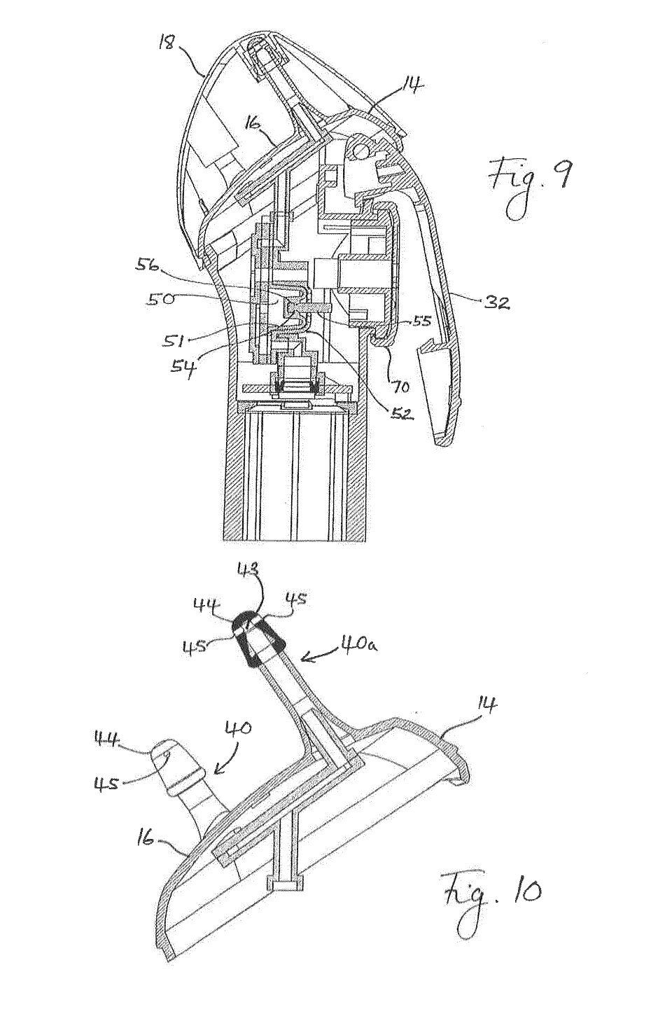

FIG. 12 is a perspective view of a pump assembly for switchable nozzle scalp applicator;

FIG. 13 is a perspective view of a pump assembly for a single or triple nozzle scalp applicator;

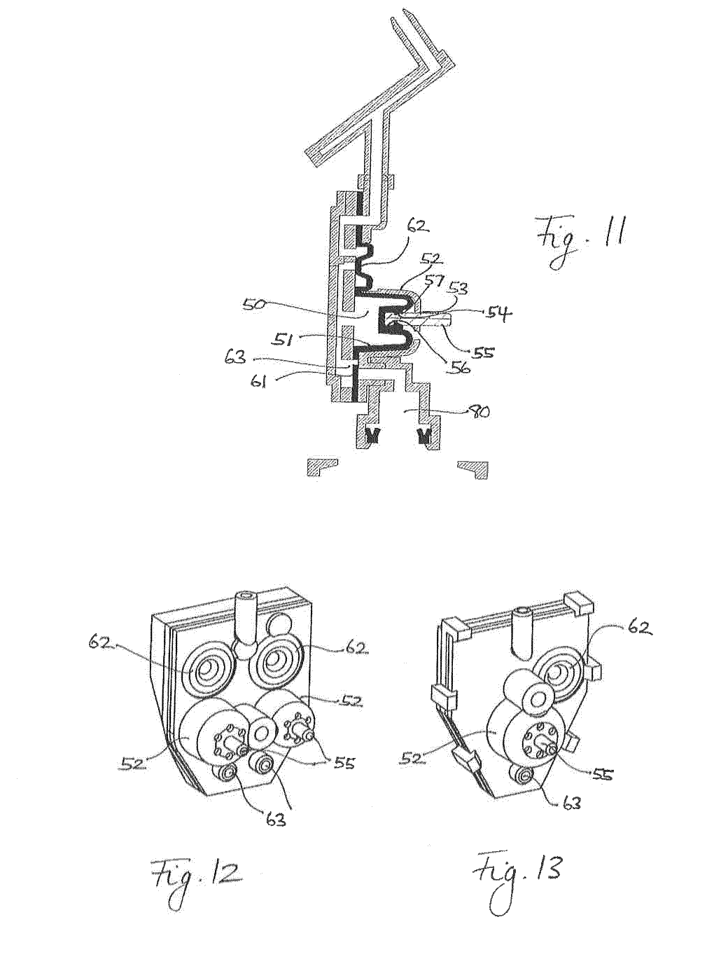

FIG. 14 is an exploded perspective view of a switching mechanism for a switchable triple nozzle scalp applicator;

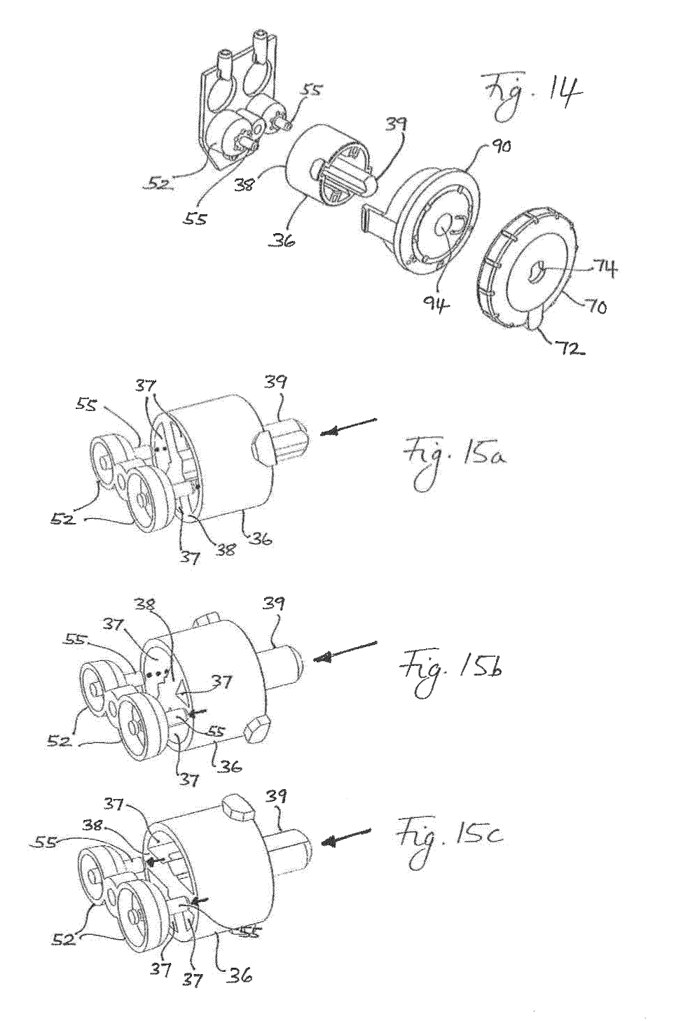

FIGS. 15a to 15c show respectively the locked position, single nozzle pump position and triple nozzle pump position of the switching mechanism of FIG. 14;

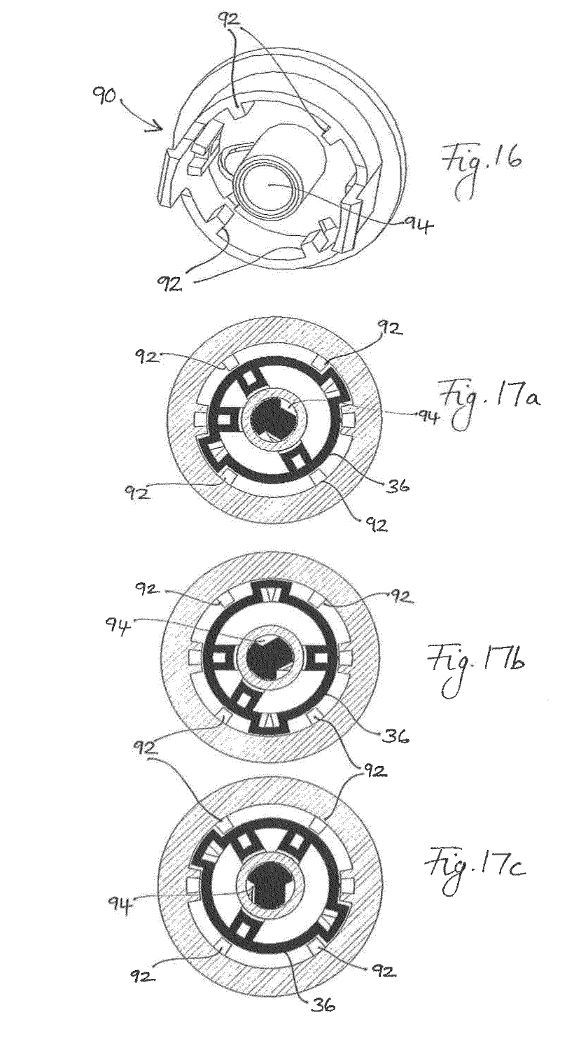

FIG. 16 is a perspective view from behind of the insert of the switching mechanism of FIG. 14; and

FIGS. 17a to 17c are cross-sections through the plunger body and insert of the switching mechanism of FIG. 14 in various relative positions.

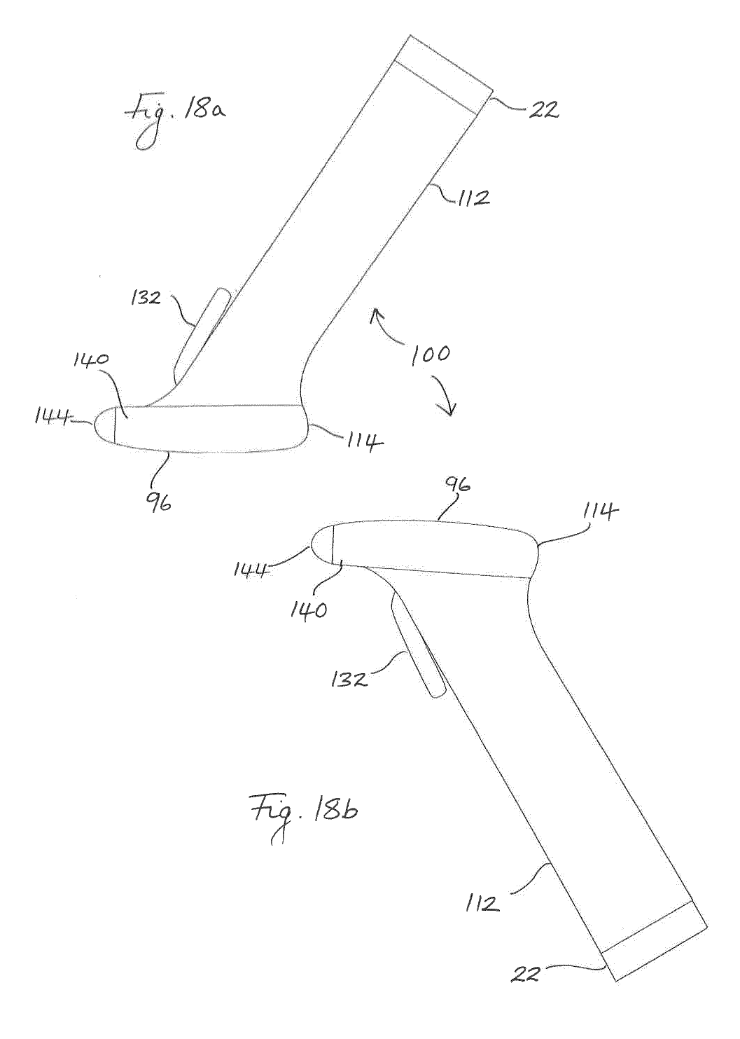

FIGS. 18a and 18b are views of an alternative single nozzle applicator for scalp and other body area application; FIG. 18c is a view of a similar fully disposable applicator;

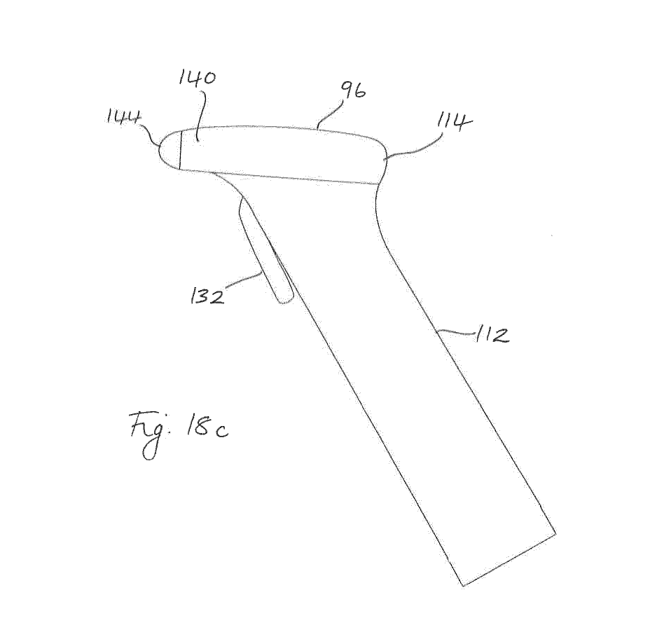

FIG. 19 is a partial cross-section of the applicator head, pump assembly and actuator of the applicator of FIGS. 18a and 18b;

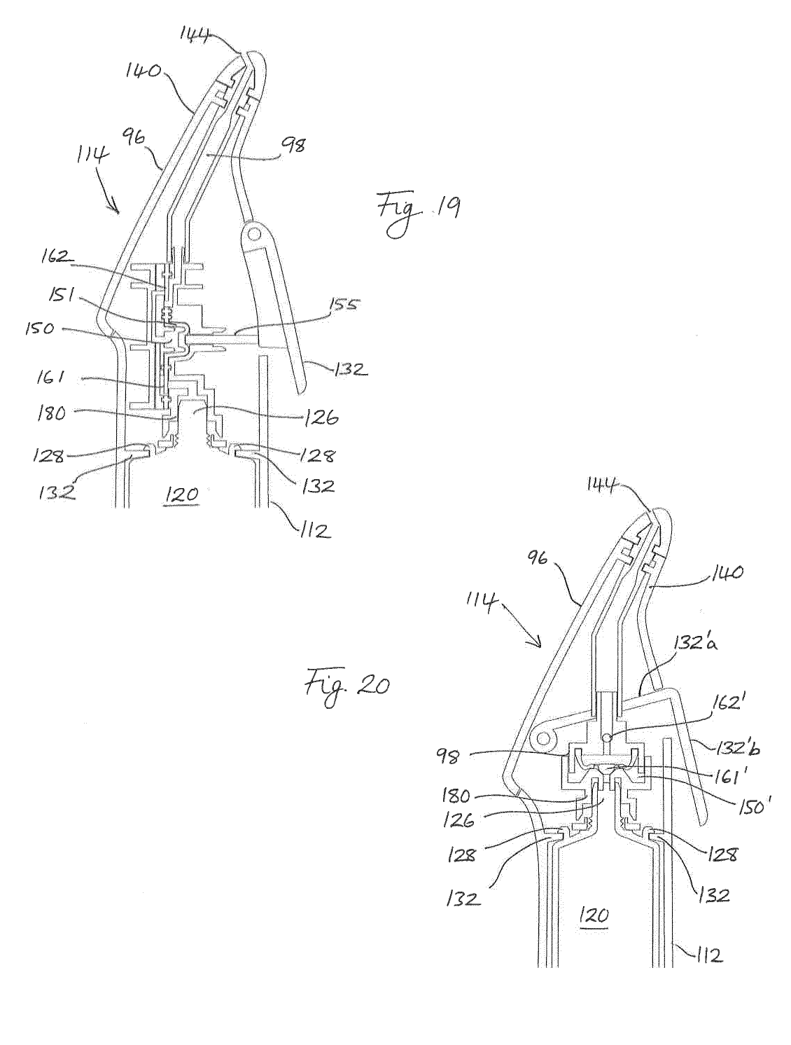

FIG. 20 is a partial cross-section of the applicator head of the applicator of FIGS. 18a and 18b fitted with an alternative pump assembly and actuator;

FIG. 21 is a perspective view of a single nozzle applicator having a first configuration;

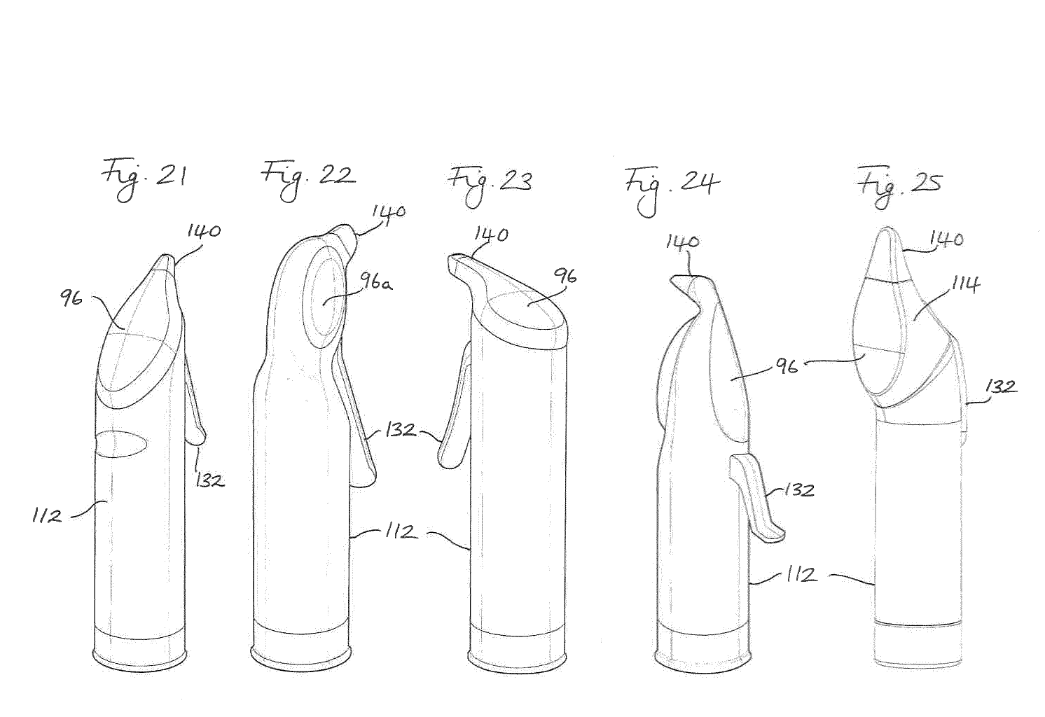

FIG. 22 is a perspective view of a single nozzle applicator having a second configuration;

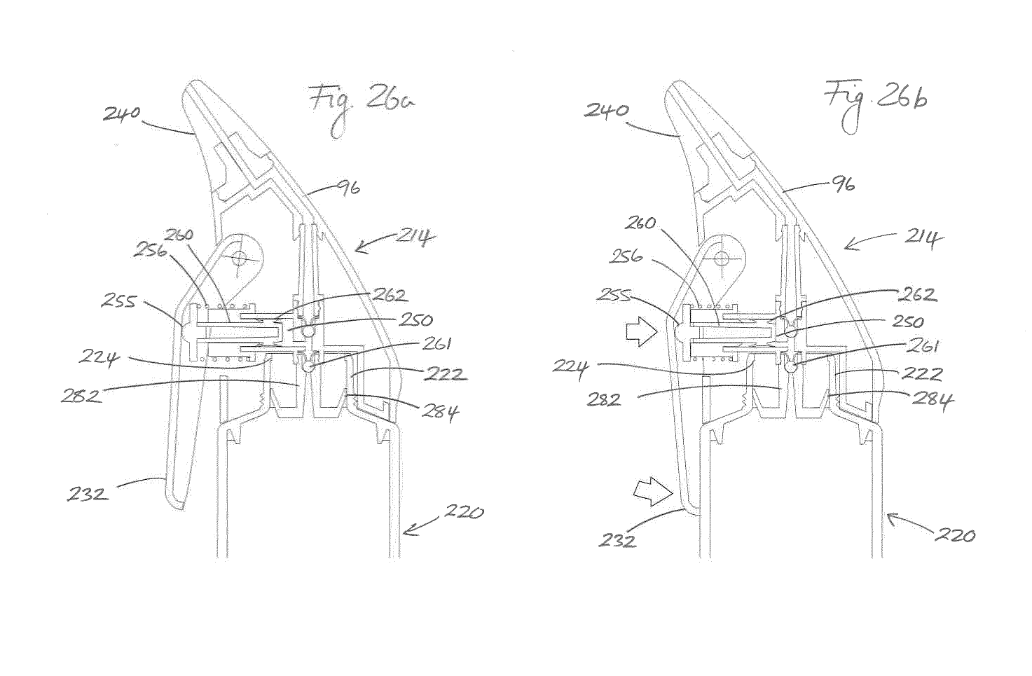

FIG. 23 is a perspective views of a single nozzle applicator having a third configuration;

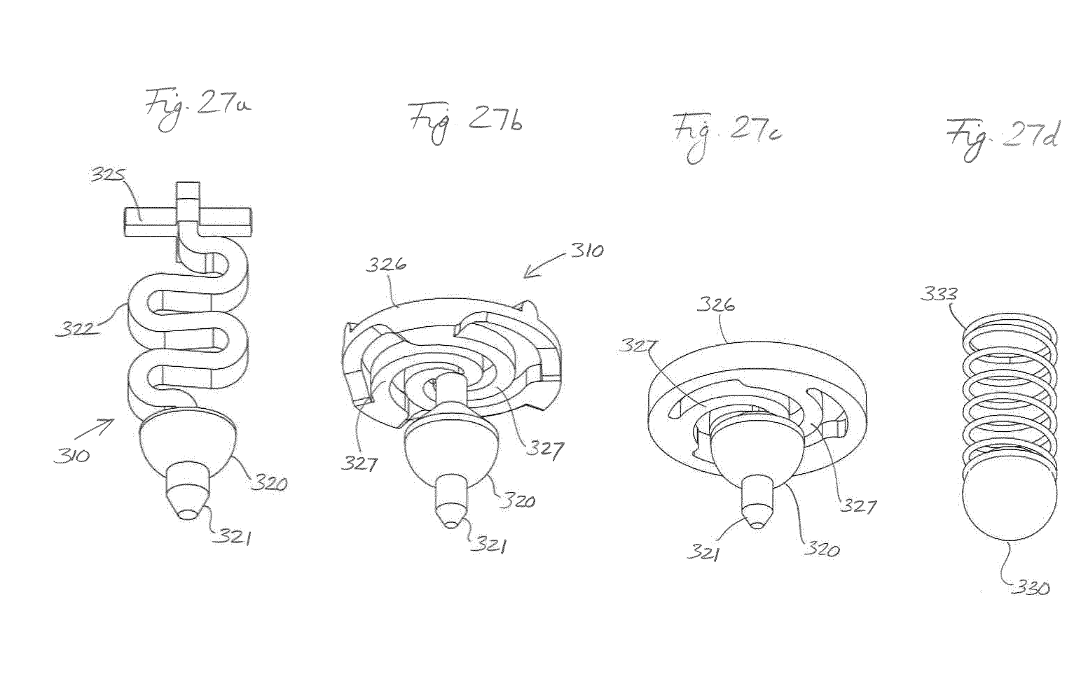

FIG. 24 is a perspective view of a single nozzle applicator having a fourth configuration;

FIG. 25 is a perspective view of a single nozzle applicator having a fifth configuration;

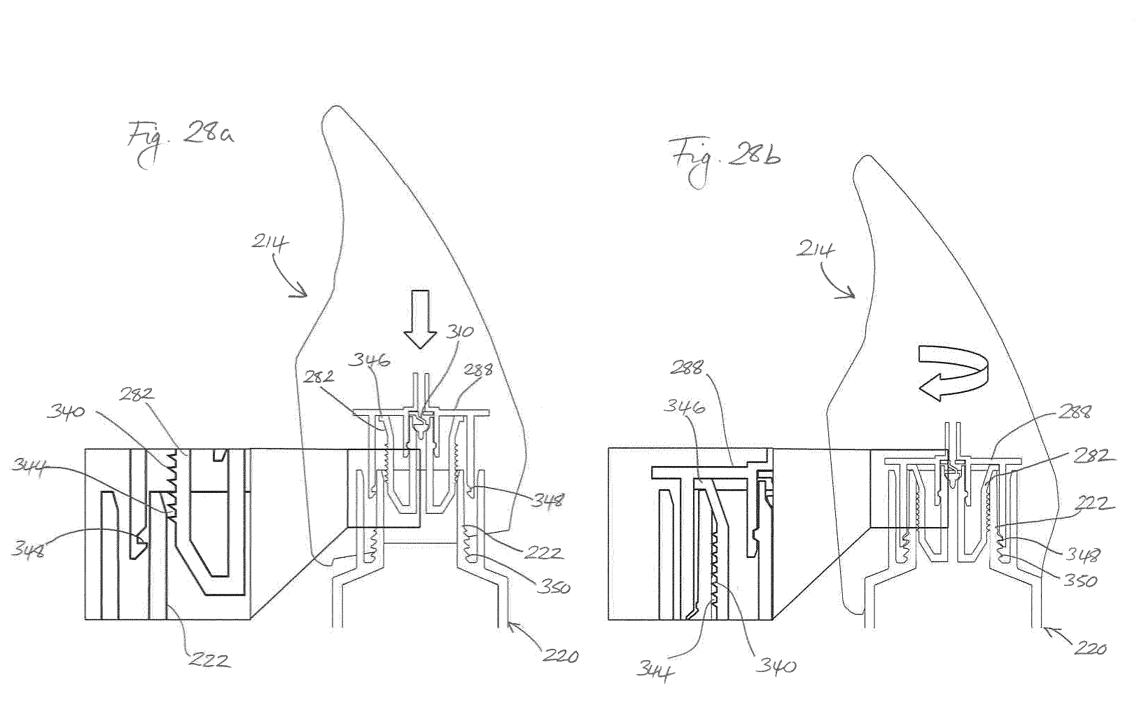

FIGS. 26a and 26b are partial cross-sections of a fully disposable applicator showing first and second lever actuator positions;

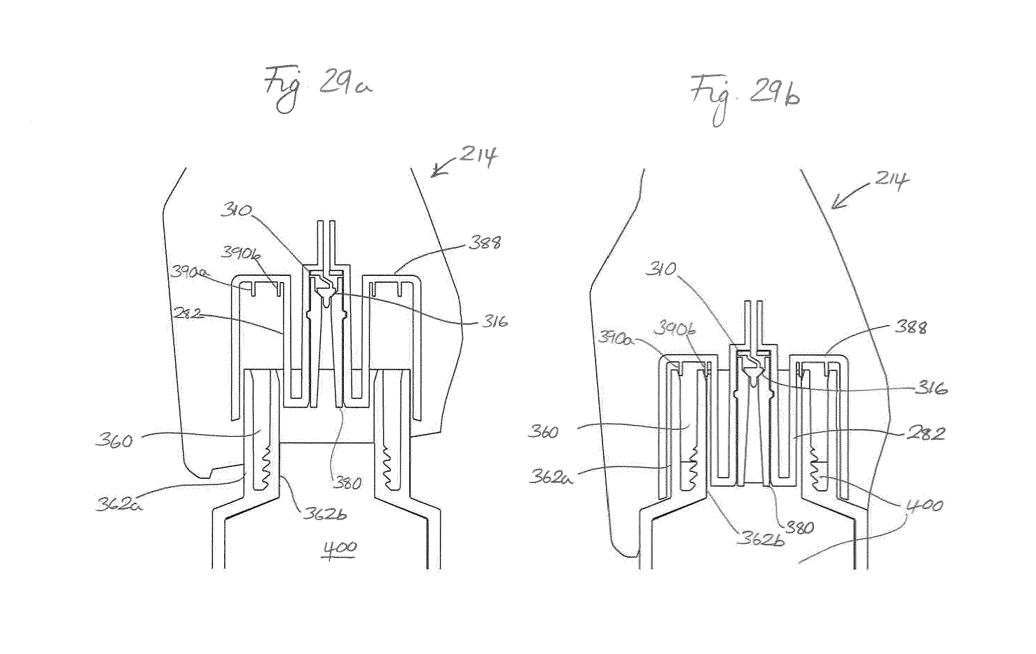

FIGS. 27a to 27d are perspective views of alternative valves for use in an applicator according to the invention;

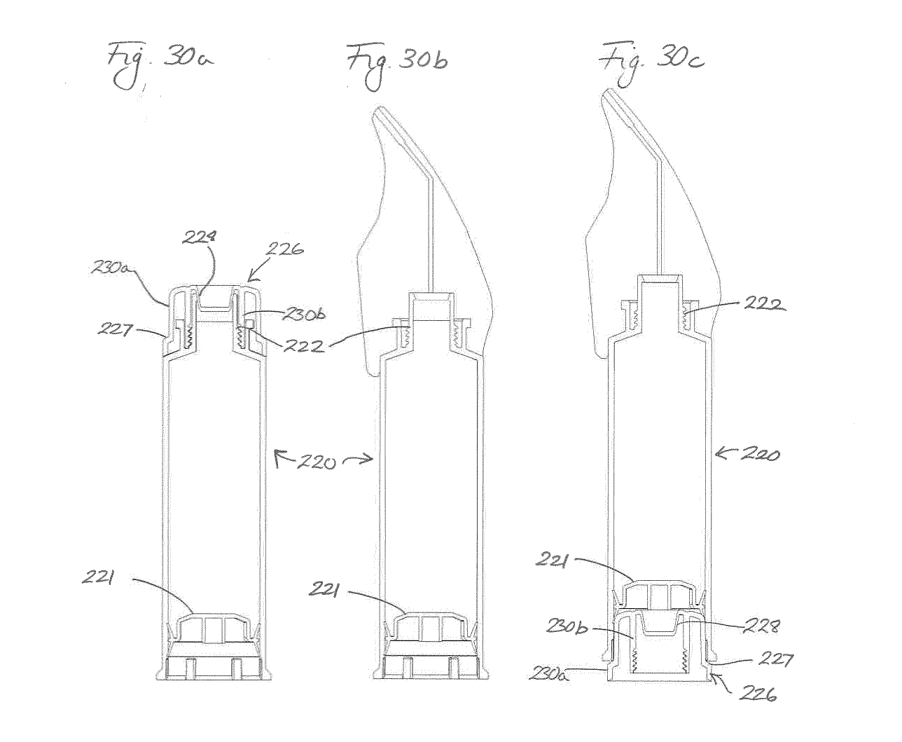

FIGS. 28a and 28b are schematic partial cross-sections through a head assembly and cartridge illustrating a first priming arrangement;

FIGS. 29a and 29b are schematic partial cross-sections through a head assembly and cartridge illustrating an alternative priming arrangement;

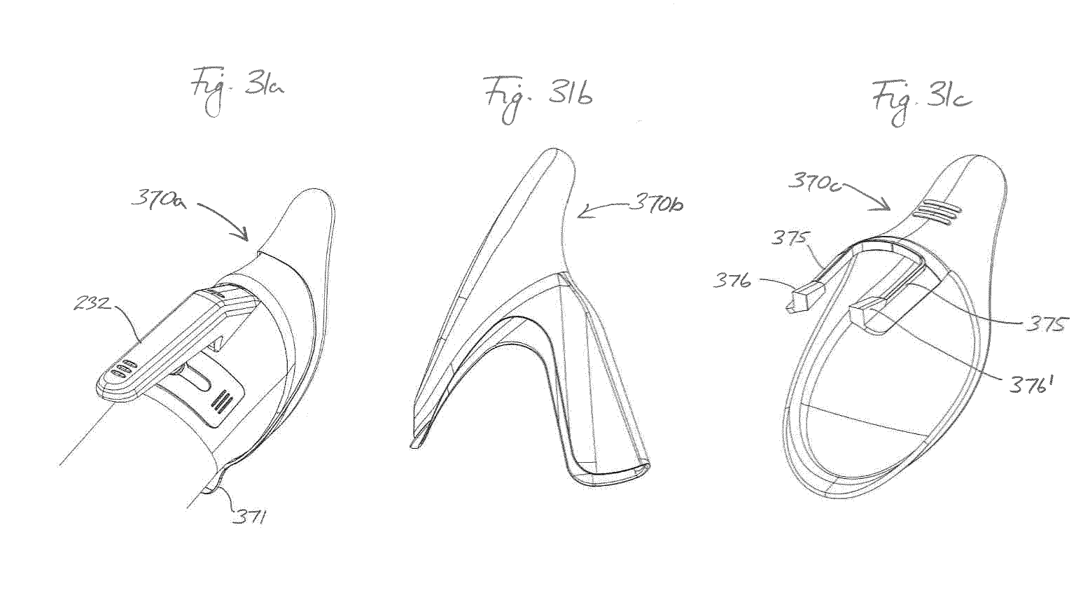

FIGS. 30a to 30c are schematic cross-sections through a cartridge with cartridge cap for use in a further priming arrangement;

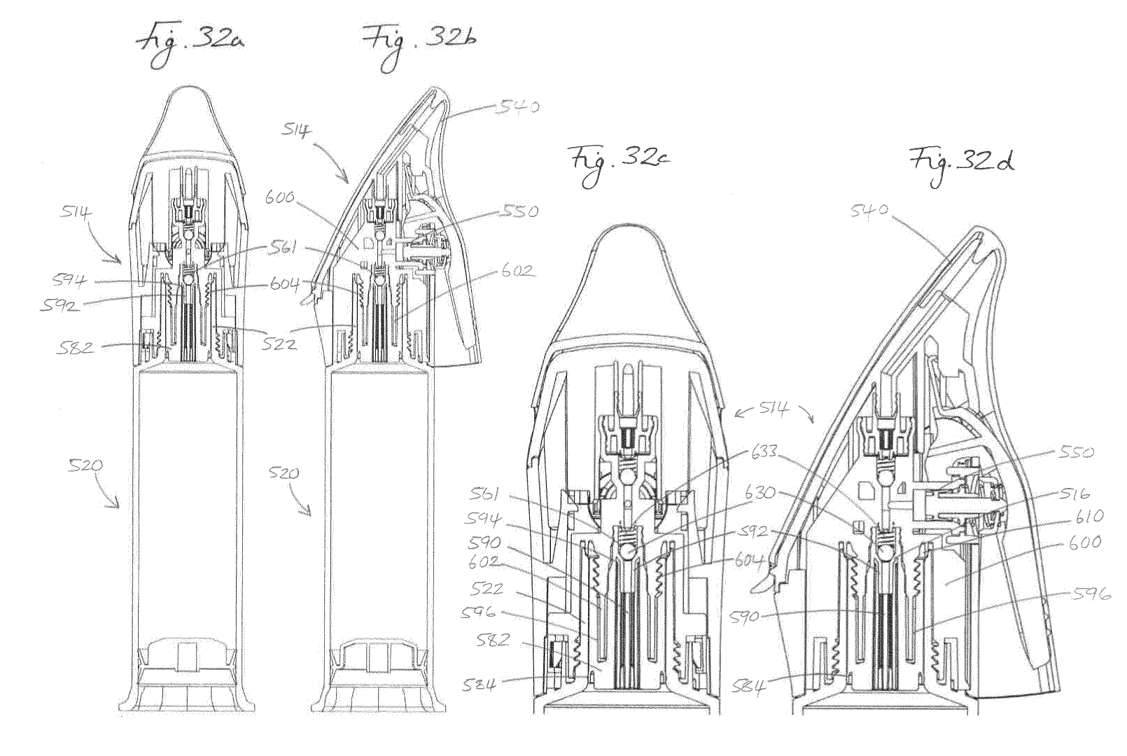

FIGS. 31a to 31c are perspective views of alternative covers for use with a single nozzle applicator according to the invention;

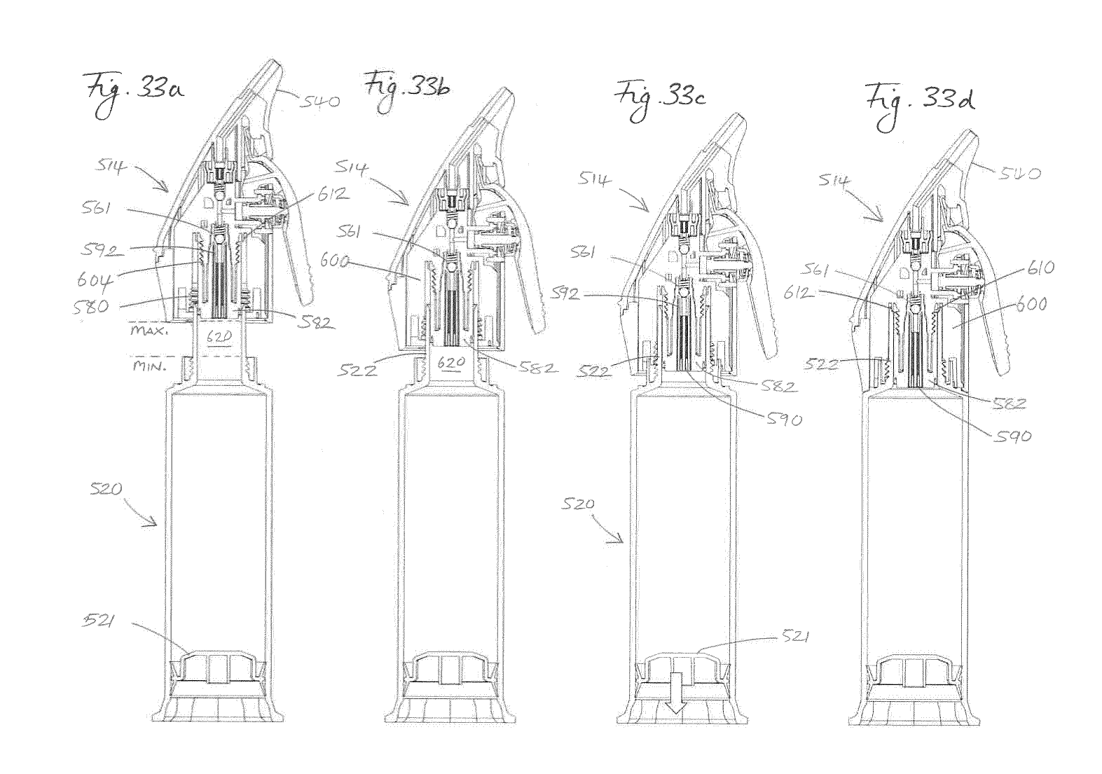

FIGS. 32a to 32d are cross-section views of a further alternative applicator incorporating a dispenser system according to another aspect of the invention;

FIGS. 33a to 33d illustrate the assembly sequence of the applicator/dispenser head and cartridge of the applicator of FIGS. 32a-32d;

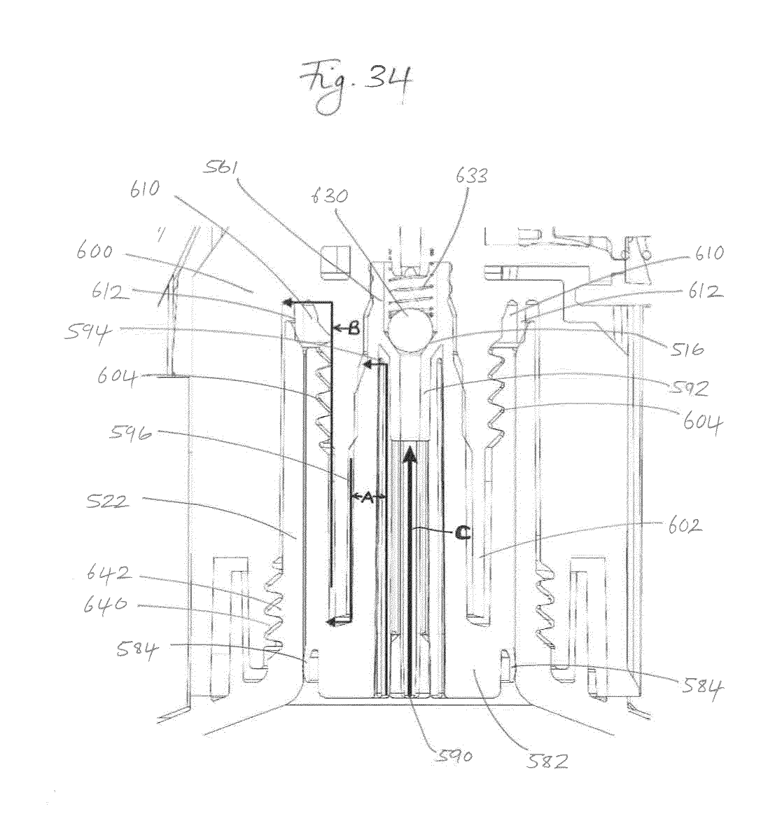

FIG. 34 is a partial enlarged cross-section from FIG. 32d illustrating the flow path for the displaced air and preparation;

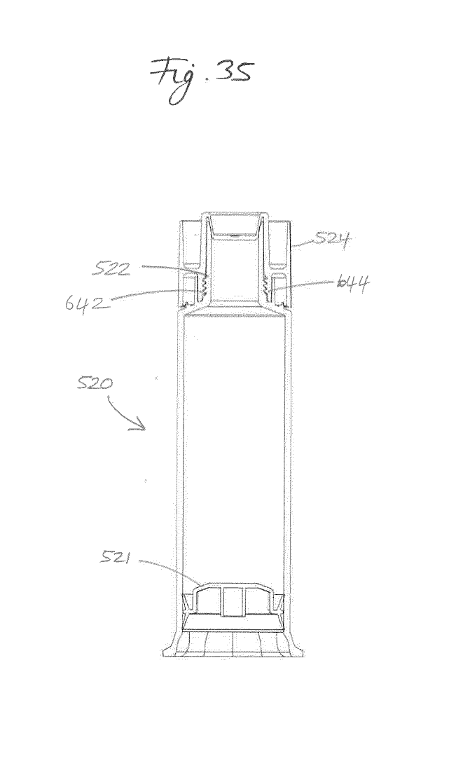

FIG. 35 is a cross-section view of a cartridge with cap for use with the applicator/dispenser of FIGS. 32a-32d;

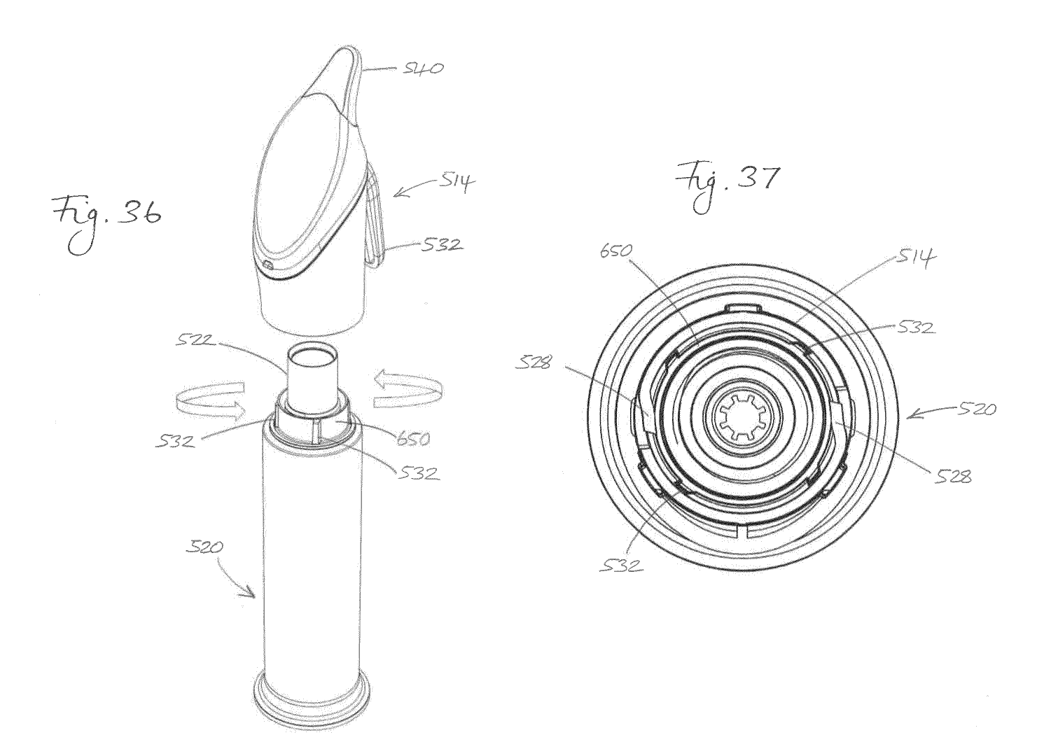

FIG. 36 is a perspective view of an applicator/dispenser head and cartridge of the applicator/dispenser of FIGS. 32a-32d illustrating a one-way locking arrangement; and

FIG. 37 is a cross-section of through the assembled applicator/dispenser of FIG. 36 in a locked configuration.

In FIGS. 1a to 1d there is shown an applicator 10 having an applicator body 12 and applicator head 14. The applicator body 12 is closed at its lower end by an end cap 22 that is part of the cartridge 20 fitted on the applicator. FIG. 2 shows the open end of the applicator body 12 into which the cartridge 20 is inserted.

A single nozzle 40 comprising a nozzle stem 42 terminating in nozzle tip 44 of softer material than the stem projects from an inclined applicator face 16 on the applicator head 14. The nozzle stem 42 tapers outwards where it joins the applicator face 16 to provide strength and a smooth contour for ease of cleaning. The nozzle tip 44 has a pair of opposite side ports 45 through which medicament is dispensed to the scalp.

The applicator 10 includes an actuator 30 in the form of lever arm 32 and actuator rod 39 which can be pushed in by the lever arm 32 to press a plunger 34 (not visible) within the applicator body 12 against a pump chamber 50 (not visible) of the pump assembly. The applicator 10 has a dial 70 with a tab 72 which can interlock with a recess on the rear side of the lever arm 32 at its distal end for preventing accidental actuation such as during storage or transport of the applicator. The dial 70 is rotatable about a central axis from the locked position to an unlocked position as shown in FIG. 1c in which an actuator rod 39 can be pushed in by the pressing the lever arm 32 towards the applicator body 12 to dispense a dose of medicament from the nozzle 40.

FIGS. 3a to 3e show an alternative applicator 10 having an enlarged applicator head 14 as compared to the applicator of FIGS. 1 and 2, this time with three nozzles 40, 40a extending from the applicator face 16. FIG. 4 shows the applicator 10 without the cartridge 20 fitted. The nozzles 40, 40a are disposed on the applicator face 16 in a triangular arrangement with the uppermost nozzle 40a on the applicator face 16 having a nozzle stem length greater than the other two, lower nozzles 40 which are of equal length. The nozzle tips 44 of all three nozzles lie in the same plane so that they can all be in contact with the scalp of a user when medicament is dispensed through all three nozzles 40 simultaneously. When the applicator 10 is held at a different angle with respect to the scalp, it is possible for the longer nozzle 40a only to contact the scalp, the latter orientation being suitable for dispensing only from the longer nozzle 40a.

The applicator 10 also has a lever arm 32 and actuator rod 39 which can be pushed in by the lever arm 32 to press a plunger 34 (not visible) within the applicator body 12 against a pump chamber 50 (not visible) of the pump assembly. Similarly, there is a dial 70 with a tab 72 for interlocking with a recess on the rear side of the lever arm 32 to guard against accidental dispensing of medicament. The dial 70 is rotatable about a central axis from the locked position as shown in FIG. 3d to a first unlocked position as shown in FIG. 3e in which an actuator rod 39 can be pushed in by pressing the lever arm 32 towards the applicator body 12 to dispense a dose of medicament from all the nozzles 40, 40a, and further rotated to a second unlocked position (not shown) in which the actuator rod 39 operates to dispense medicament from the longer nozzle 40a only.

FIGS. 5a to 5c show a switchable applicator 10 fitted with a cap 18 to protect the nozzles 40, 40a of the applicator when not in use, such as for storage or transportation. The cap 18 has a thickened rim (not shown) that is caught and held by a corresponding recess (not shown) around the perimeter of the applicator head 14 to retain the cap 18 in place. The cap 18 is made of a flexible material to enable the cap to be prised away from the applicator head 14 when preparing the applicator 10 for use. A series of ribs 19 on opposite sides of the cap 18 provide a gripping function to assist a user when removing of the cap.

FIGS. 6a to 6c show a cartridge 20 for use in all variations of scalp applicator 10, including those of FIGS. 1 and 3. The cartridge 20 is supplied with an cartridge cap 24 or top that is removed prior to fitting in the applicator 10.

FIGS. 7a to 7c correspond to the FIGS. 6a to 6c with the cap 24 removed. The dispensing or outlet end 26 of the cartridge 20 includes a locking flange 28 that interlocks with a corresponding recess near the cartridge port 80 of the applicator as will be seen from FIG. 8. The cartridge 20 can thus be fitted to the applicator 10 by first removing the cartridge cap 24, inserting the cartridge 20 so that outlet end 26 enters the cartridge port 80 and the flange 28 abuts a shoulder 82 of the cartridge port 80 to indicate it is fully inserted whereupon the cartridge 20 is twisted through 90° so that the locking flange 28 is in interlocking relationship with the recess adjacent the cartridge port 80.

FIG. 8 shows the cartridge 20 fitted to the applicator 10 and, for simplicity, does not show the pump assembly nor the dial, etc. The end cap 22 of the cartridge 20 abuts the open end of the applicator body 12 when fully inserted, effectively creating a continuous sealed external surface which may be readily wiped to maintain the applicator in a clean, hygienic condition.

FIG. 9 provides a more detailed cross-section of the applicator 10 of FIGS. 3a to 3e in the region of the applicator head 14 and upper applicator body 12. The applicator 10 is of the type having two rolling diaphragms 51 of differing volumes (only one visible) for pump chambers 50, each diaphragm 51 being contained within an appropriately sized casing 52 having a central aperture 53 through which a respective chamber pin 55 projects. The chamber pin 55 is retained in an outwardly directed recess 56 on its respective diaphragm 51 which has a rib 57 that engages with a groove 54 on the chamber pin 55.

An outlet 66 for medicament discharged from the diaphragm 51 visible in FIG. 9 (but shown in more detail in FIG. 11) leads only to the uppermost nozzle 40a, that is the longer of the three nozzles on the applicator head 14.

FIG. 10 provides more detail of the nozzle construction and fluid pathway that leads from the pump chamber 50 (the diaphragm 51). The nozzle stem 42 has an end port 43 and a nozzle tip 44 having opposing side ports 45 is fitted over the end of the nozzle stem 42. The nozzle tip 44 which is of a resilient material is retained on the nozzle stem 42 by stretching over the end of the stem whereupon an internal rib on the inner rim of the tip 44 springs into a corresponding groove around the stem end.

As shown in FIG. 11, the rolling diaphragms 51, flap inlet valves 61 and outlet valves 62 can be provided as one unit, by molding an elastomeric material to provide the requisite shapes of the diaphragms and outlet valves and subsequently punching the material to provide the flap inlet valve 61.

FIG. 12 is a perspective view of part of a pump assembly adapted for a switchable applicator 10, specifically switchable between single and triple nozzle dispensing. Dual inlets 63 provide fluid pathways to two rolling diaphragms 51 (not visible) contained in respective casings 52 having chamber pins 55 extending therethrough. Elastically deformable outlet valves 62 are provided for each diaphragm chamber 50.

FIG. 13 shows an equivalent part to that of FIG. 12, but intended for use in a single nozzle or a triple nozzle applicator 10. Only one rolling diaphragm 51 (not visible) is provided, the capacity of which being different depending on whether the applicator 10 is a single nozzle or triple nozzle applicator. The diaphragm 51 is contained in an associated casing 52, with a single inlet 63 that receives medicament from the cartridge 20, a deformable outlet valve 62 and a single outlet leading to the sole nozzle 40a.

FIG. 14 is an exploded view of a switching (nozzle selector) mechanism for incorporating in an applicator 10 that may operate in either single nozzle or triple nozzle dispensing mode depending on the selection made.

The pump manifold 64 has dual inlets 63, first and second rolling diaphragm chambers 51 (not visible) each having an associated casing 52 and chamber pin 55. The chamber that feeds through to the single nozzle 40a has half the capacity of the other chamber that feeds through to the remaining two nozzles 40, consequently the casings 52 are also of different sizes. However the respective chamber pins 55 project to the same plane which lies parallel to the front face 38 of plunger body 36.

A rotatable plunger body 36 is provided with spaced apart apertures 37 on its front face 38 which apertures 37 can accommodate one or both the chamber pins 55 according the mode selected when the plunger body 36 is pushed in. An actuator rod 39 extends from the rotatable plunger body 36 and projects beyond the rear of the plunger body. The actuator rod 39 has an asymmetric cross-section that slots through a similarly shaped asymmetric aperture 74 in the dial 70. An insert 90 between the plunger body 36 and dial 70 has internal projections 92 that act to prevent the actuator rod 39 from being moved back and forth unless the plunger body 36 is in one of the correct positions.

As shown in FIG. 15a, if the plunger body 36 is in the “locked” position, when the actuator rod 39 is pushed, both chamber pins 55 go into the apertures 37 in the plunger body 36 and so the pins 55 are not pushed. Since it is the pins 55 that cause collapse of their respective rolling diaphragm 51, nothing is dispensed from this position.

FIG. 15b shows the plunger body 36 in the single nozzle 40a dispensing position, whereby when the actuator rod 39 is pushed, one of the pump pins 55 goes into the aperture 37 in the plunger body 36 and the other pump pin 55 is pushed by the front face 38 of the plunger body 36, thereby discharging medicament from one pump chamber, notably the pump chamber that feeds the single nozzle 40a.

FIG. 15b shows the plunger body 36 in the triple nozzle dispensing position, whereby when the actuator rod 39 is pushed, both pump pins 55 are pushed by the front face 38 of the plunger body 36, thereby discharging medicament from both pump chambers 50 and resulting in medicament being dispensed from all three nozzles 40a, 40.

FIG. 16 shows in more detail features of the insert 90 of FIG. 14, notably four equidistant internal projections 92 that serve as keys for the plunger body 36, permitting advancement of the plunger body 36/actuator rod 39 only when the plunger body 36 is in a locked (non-dispensing) position or in single or triple nozzle dispensing positions. The insert 90 has a central round aperture 94 through which the actuator rod 39 may pass.

The various relative positions of the plunger body 36 and insert 90 that permit triple nozzle pumping, no pumping, and single nozzle pumping are shown sequentially in FIGS. 17a to 17c. As will be apparent from FIG. 17b, the keys 92 of the insert do permit a slight amount of movement either side of the locked position to rotate the dial 70 and hence release the lever arm 32 if interlocked with the tab 72 of the dial.

FIGS. 18a and 18b illustrate an alternative single nozzle applicator 100 for dual purpose use, namely for use in applying medicament to body areas as shown by the orientation of the applicator in FIG. 18a and for use in applying medicament to the scalp as shown by the orientation in FIG. 18b. The applicator 100 includes an applicator body 112 closed at its lower end by end cap 22 that is part of the replaceable medicament cartridge 120. The applicator head 114 has a tapered nozzle 140 extending from and continuous with gently convex application face 96, inclined with respect to the longitudinal axis of the applicator body, which face is used to spread dispensed medicament over body areas other than the scalp. Medicament is dispensed by squeezing lever arm 132 against the applicator body 112 which action presses a plunger (not visible) within the pump chamber (not visible) to force medicament through the nozzle 140 and out through the nozzle tip 144 made of resilient material. When used to apply medicament to the scalp, the nozzle tip 144 is gently pushed towards the scalp, parting the hair as it is advanced, and the lever arm 132 is squeezed once the tip is resting on the scalp to dispense the medicament directly on the scalp from the nozzle outlet. The nozzle tip 144 is then used to spread the dispensed medicament over the adjacent scalp area. When only a small body area is to be treated, the nozzle tip 144 can equally be used to spread dispensed medicament over that area rather than using the application face 96.

FIG. 18c shows an applicator similar to that of FIGS. 18a and 18b but being fully disposable. In other words, the applicator is not intended to be used with replaceable cartridge, rather the entire device will be disposed of once the medicament reservoir is depleted.

A partial cross-section through the applicator head 114, pump assembly and actuator of the applicator of FIGS. 18a and 18b is provided at FIG. 19. The dispensing end 126 of cartridge 120 is fluidly connected to the applicator head 114 via cartridge port 180 and locked to the applicator by means of locking tabs 128 that interlock with projections 132 on the applicator body when the cartridge is inserted and then rotated. The cartridge port 180 leads to a one-way inlet valve 161 through which medicament from the cartridge 120 is permitted to flow to pump chamber 150. The chamber 150 has a rolling diaphragm (sometimes referred to as a membrane pump) 151 operable upon by chamber pin 155, the pin being advanced to reduce the volume of the chamber 150 when the lever arm 132 is squeezed. As the chamber pin is advanced, medicament is prevented from being forced back into the cartridge by inlet valve 161 and instead is forced out of the chamber 150 through outlet valve 162 and into the bore 98 of nozzle 140 from where it is discharged through nozzle tip 144 onto the scalp or skin of the user.

The lever arm 132 pivots about a fulcrum within the applicator head 114 behind the nozzle 140, the arm extending away from the applicator body 112 beneath the nozzle 140 in the manner of a trigger.

Instead of a membrane pump as shown in FIG. 19, the applicator may include a vertical diaphragm pump as in FIG. 20. In this arrangement, the lever arm 132′ is substantially L-shaped having a first arm 132′a which is substantially transverse to the longitudinal axis of the applicator body 112 and a second arm 132′b which depends substantially at right angles to the first arm. The lever arm 132′ pivots about a fulcrum within the applicator head 114 adjacent the rear of the application face 96, such that on squeezing the second arm 132′b towards the applicator body 112, the first arm 132′a is brought down against the diaphragm pump 98 which in turn is driven down to reduce the volume of the pump chamber 150′. The resulting increased pressure in the pump chamber prevents one-way inlet valve 161′ from opening, but does result in outlet valve 162′ opening to discharge medicament into the bore of the nozzle 140 from where it then discharged through the nozzle tip 144 onto the scalp or skin of the user.

The angle of inclination and orientation of the application face with respect to the applicator body and the nozzle may be altered, as may the position of the actuation lever. A number of variations in the applicator configuration are exemplified in FIGS. 21 to 25, each of which illustrate dual function applicators, adapted for both scalp and body application.

For example, FIG. 21 shows an applicator similar to that of FIGS. 18a and 18b, but the application face 96 and tapered nozzle 140 are inclined at a greater angle with respect to the applicator body 112. The underside of the tapered nozzle 140 is substantially flat whereas the upwardly directed surface of the nozzle is gently rounded and continuous with the application face 96. Whilst the lever arm 132 is shown beneath the nozzle 140, its position could equally be on the opposite side of the applicator body 112, extending from below the lower end of the application face 96.

In FIG. 22, the applicator head 114 has a pair of opposing, outwardly (side) facing application faces 96a, 96b between which a tapered nozzle 140 extends at an upwardly inclined angle with respect to the applicator body 112.

The applicator of FIG. 23 is more akin to that of FIG. 21 except the angle at which the application face 96 and nozzle 140 extends is less steeply inclined and the application face 96 is substantially round as opposed to oval. In addition, while the transition between the application face 96 and nozzle 140 is still a smooth one, the nozzle is angled down with respect to the application face.

In each of the illustrated variations of FIGS. 21 to 23 the lever 132 is located beneath the nozzle 140. However, FIG. 24 shows further variation in which the lever 132 is not only oppositely directed to the nozzle 140, but the nozzle 140 substantially overhangs the application face 96. In this variant, the underside of the nozzle 140 is substantially flat whereas the upper face is rounded.

In FIG. 25, the applicator head 114 is off-set with respect to the applicator body 112, and the application face 96 and nozzle 140 extend substantially parallel to the longitudinal axis of the applicator body. The application face 96 and underside of the nozzle together present a continuous surface for spreading medicament over a body area.

FIGS. 26a and 26b show partial cross-sections of a fully disposable applicator with head assembly 214 fitted to cartridge 220 in accordance with a further aspect of the invention. The cartridge port of the head assembly 214 includes a stopper 282 having a lip seal 284 that seals against the inner wall of the cartridge neck 222. The upper, discharging end of the cartridge neck 222 has an inwardly directed flange 224 which prevents the lip seal 284 from passing back over the flange and hence separation of the cartridge neck 222 from the head assembly 214 once the cartridge 220 has been inserted. The head assembly 214 includes an actuator including a horizontal force pump having a piston 260 provided with a lip seal 262 in sealing contact with the inner cylinder wall of the pump chamber 250. The trailing end of the piston 260 carries a button 255 that is acted upon by the lever arm 232 of the actuator driving the piston 260 along the cylinder against a resilient force provided by the spring 256. FIG. 26a shows the head assembly 214 prior to actuation by the actuator lever 232 and in which position the pump chamber 250 is charged with medicament from the cartridge 220. When the lever 232 is depressed, such as by squeezing towards the cartridge, the piston 260 is advanced through the pump chamber 250 to dispense medicament as illustrated in FIG. 26b. In particular, the inlet valve 261 is kept closed due to the pressure of the medicament being evacuated from the pump chamber 250 but the outlet valve 263 is forced open to allow the medicament to pass through to the nozzle 240 whereupon a dose of the medicament is discharged from the nozzle tip.

FIGS. 27a to 27d are perspective views of alternative valves for use in an applicator according to the invention, such as in the applicator as shown in FIGS. 26a and 26b. Each of the valves of FIGS. 27a to 27c comprises a valve member, spring and anchor portion, whereas the valve of FIG. 27d comprises simply a ball 330 and spring 333. In FIG. 27a there is shown a unitary valve moulding 310 comprising a substantially hemispherical valve member 320 that in use is urged by the spring portion 322 against a valve seat (not shown). The valve member 320 includes an extension portion 321 that remains captive in the fluid pathway ensuring that the valve member does not become misaligned with respect to the valve seat. The valve of FIG. 27a includes an anchor portion 325 which is generally cruciform, the outer portions of each arm capable of being captured between opposing surfaces in the cartridge port or pump chamber. In FIG. 27b, in which the valve is again a unitary moulding 310, instead of the spring portion depending from the anchor portion, the valve member 320 is suspended on resilient arms 327 that generally extend from and lie parallel to an annular anchor portion 326. When a force acts on the valve member 320, such as when medicament is forced out from the pump chamber (in the case of an outlet valve) or when medicament is drawn from the cartridge to the pump chamber (in the case of an inlet valve), the valve member is urged up against the returning force exerted by the resilient arms 327 to open the valve. The valve of FIG. 27c is similar to that of FIG. 27b, but is not a unitary moulding.

FIGS. 28a and 28b are schematic cross-sections through a head assembly 214 and cartridge 220 illustrating a first priming arrangement in which air is bled out through the interface between the head assembly and cartridge. The head assembly 214 includes a stopper 282 attached to a cap 288, and a unitary valve moulding 310 of the type generally shown in FIGS. 27a and 27b anchored therebetween. The stopper 282 has an external helical lip seal 340 that allows air to escape as the stopper is pushed into the cartridge neck 222 during fitting of the cartridge 220 to the head assembly 214. As the head assembly 214 becomes fully engaged with the cartridge 220, the stopper 282 applies pressure to the medicament in the cartridge once all the air has been evacuated. As medicament enters the thread-like channel 344 created by the helical seal 340, the resulting back pressure serves to push the cartridge plunger (not shown) back down the cartridge body, avoiding overspill of medicament. In order to lock and seal the head assembly 214 to the cartridge 220, the cartridge is twisted at least a couple of turns to compress the flange seal 346 on the stopper 282 between the upper end surface of the cartridge neck portion and the cartridge cap member 288 as the groove 348 on the cap member 288 engages the outer screw thread 350 of the cartridge neck portion.

An alternative priming arrangement is shown in FIGS. 29a and 29b in which the chamber 360 created between the double walls 362a, 362b of the cartridge neck 222 are made use of to receive any medicament overspill as the stopper 282 is advanced into the neck. In this arrangement, the stopper 282 includes a cap portion 388, as opposed to a separate cap member, that provides a seal with the outer neck wall 362a. The cap portion 388 has two concentric walls 390a, 390b for sealing against the inner and outer neck walls 362a, 362b respectively once the stopper 282 has been fully advanced, isolating any medicament 400 that overspills into the chamber 360. In this arrangement, an insert 380 captured within the stopper 282 serves to anchor the inlet valve 310 and provide the valve seat 316. The head assembly 214 is fitted to the cartridge 220 in a similar manner to the arrangement shown in FIGS. 28a and 28b, namely by initially pushing the head assembly onto the cartridge and then by twisting the cartridge to lock and seal the head to the cartridge.

Another priming arrangement is shown in FIGS. 30a to 30c which are schematic cross-sections through a cartridge 220 having cartridge cap 226 that doubles as a priming insert. FIG. 30a shows the double-walled cartridge cap 226 screwed on to the neck of the cartridge 220 as it would be during storage. The cap 226 includes a recessed portion 228 extending into the cartridge neck 222 limiting the amount of air that is trapped in the cartridge 220 after filling and securing the cap. The outer wall 230a of the cap 226 is contoured and lies flush with the cartridge outer wall at its open end. The inner wall 230b of the cartridge cap 226 has a threaded portion engaged with a complementary threaded portion on the inner wall of the cartridge neck.

FIG. 30b shows the cartridge 220 fitted to the applicator head assembly 214 after the cap 226 has been removed. The assembled applicator still includes a volume of air at the neck of the cartridge that must be discharged before the applicator can dispense a medicament dose. FIG. 30c shows how the applicator is primed for use by insertion of the cartridge cap 226, smaller circumference first, into the non-discharging end of the cartridge. Advancing the cap 226 into the rear end of the cartridge 220 pushes the cartridge plunger 221 further into the cartridge thereby displacing the remaining air at the neck of the cartridge. The extent to which the priming insert/cap 226 can be inserted is limited by the shoulder portion 227 created by the different cap outer diameters. Thus, the cap 226 can generally be inserted sufficiently to expel all air from the assembled applicator. After priming, the cap/insert 226 can be removed from the rear end of the cartridge 220 and discarded, or left in place as desired.

FIGS. 31a to 31c are perspective views of alternative covers 370a, 370b, 370c for use with a single nozzle applicator. In FIG. 31a, the cover 370a shields the nozzle and application face of the applicator maintaining the applicator in a hygienic condition ready for use. The cover 370a includes a tab 371 that a user may press or pull when attaching or detaching the cover from the applicator. The cover 370a may have a projection (not shown) on its underside near the tab 371 to engage in a corresponding groove in the applicator head to prevent the cover from otherwise falling off.

The cover of FIG. 31b has a similar profile to that of FIG. 31a except that it extends to cover the actuator lever 232 to prevent accidental actuation when the applicator is not in use and also to protect the actuator lever from possible damage, such as may occur if the lever catches on anything.

Instead of shielding the actuator lever, the cover of FIG. 31c includes a pair of opposing arms 375, 375′ each terminating in a projection 376, 376′ that wedges behind the lever when the arms are clipped around the lever 232 as the cover is installed on the applicator. The wedges 376, 376′ effectively prevent the actuator lever 232 from being depressed towards the applicator body or cartridge, thereby acting as a lever lock to prevent accidental actuation.

In FIGS. 32a to 32d, there is shown an applicator according to a further aspect of the invention, namely including a dispensing head 514 of the type having a stopper 582 for displacing air and preparation from the cartridge nozzle 522 on assembly. The stopper 582 is provided with a lip seal 584 around its insertion end to seal against the inner wall of the cartridge nozzle 522. The stopper 582 is double walled having a central bore 590 accommodating an inlet valve 561 comprising a ball 630 and spring 633. A straw or inner tube 592 extends into the bore 590 from the valve seat 516 and a vent 594 is provided through the inner wall of the bore 590 at its uppermost end, beyond the lowermost end of the inner tube 592. The vent 594 leads to a passage 596 in the form of a space in the U-shaped channel between the stopper 582 and tubular portion 602 of the manifold 600, providing a flow path for air and preparation as far as a constricted, threaded section 604 which resists the flow of preparation whilst allowing passage of air.

A chamber 610 is provided in the manifold 600 beyond the threaded section 604 and a wall 612 in the cartridge port 580 of the manifold seals against the upper end of the cartridge nozzle 522 so that air from the nozzle displaced by the stopper 582 is ultimately trapped in the chamber 610.

The dispensing head 514 of FIGS. 32a to 32d further includes a dispensing nozzle 540 and the manifold 600 also accommodates the pump chamber 550 of a pump assembly similar to that shown in FIGS. 26a and 26b. The dispensing head 514 as shown is fitted with a head cover 570 having a protrusion 572 that seals the nozzle tip to prevent oxidation of preparation that has been pumped through to the nozzle whilst the dispenser is not in use.

FIGS. 33a to 33d show the effect achieved by the stopper 582 when the applicator/dispenser head 514 is fitted into the cartridge nozzle 522 during assembly of the applicator of FIGS. 32a to 32d. Beginning at FIG. 33a, as the cartridge nozzle 522 is brought into alignment with the cartridge port 580 of the dispenser head 514, the lip seal 584 around the forward end of the stopper 582 seals against the internal wall of the nozzle 522 and the stopper reaches the preparation 620 in the nozzle. As illustrated here, the cartridge 520 has been charged with preparation 620 to the maximum fill level. Upon further insertion of the cartridge nozzle 522 into the cartridge port 580, shown in FIG. 33b, the stopper 582 displaces the preparation 620 which is forced up through the central bore 590 of the stopper 582. The diameter of the inner tube or straw 592 is restricted such that preparation 620 is forced around the tube 592 to the vent 594 at the upper end of the stopper bore 590 upstream of the inlet valve 561. Preparation 620 is forced through the vent 594 into the space between the stopper 582 and surrounding manifold wall 612 as far as the threaded constricted section 604 which resists further preparation flow. As the stopper 582 is advanced yet further, as in FIG. 33c, back pressure created due to the preparation 620 entering the constricted section 604 becomes greater than the pressure needed to move the plunger 521 in the cartridge 520 with the result that the plunger 521 is reversed along the cartridge body expanding the volume of the cartridge 520 to accommodate preparation 620 displaced from the nozzle 522 by the stopper 582. To fully insert the stopper 582, the cartridge 520 is then screwed on to the dispenser head 514 via the complementary screw threads 640, 642 around the exterior base of the nozzle wall and the manifold cartridge port 580. Once the stopper 582 is fully inserted, as shown in FIG. 33d, a wall 612 in the cartridge port 580 seals against the end of the cartridge nozzle 522 at its top edge creating a sealed system. Displaced air is trapped in the chamber 610 at the end of the constricted section 604.

The path taken by air and preparation displaced by the stopper 582 is shown in more detail in FIG. 34. Arrows A and B show the path taken by the air displaced by the stopper 582 on first assembly; preparation will also be forced along this path but only as far as the threaded section 604 at which point the passage 596 or channel becomes very small and the pressure required to push preparation 620 further through the threaded section 604 becomes higher than the pressure required to push the plunger (not shown) in the cartridge 520, so that no further preparation 620 is transferred and the remaining preparation is kept within the cartridge 520. In addition, the stopper 582 pushes preparation 620 to the bottom of the inner tube 592, indicated by Arrow C. The assembled applicator/dispenser is now primed to the extent that when a first dispensing operation occurs by squeezing lever arm 532 to advance the piston 560 in the pump chamber 550, the preparation 620 will be immediately drawn up through the inner tube 592 to wet the ball 630 of the inlet valve 561.

A cartridge 520 with cap 524 for use with the dispenser head 514 shown FIGS. 32 and 33 is illustrated in cross-section at FIG. 35. The cartridge 520 has a plunger 521 that seals against the internal cartridge wall and flares out at the base to provide stability when stood in an upright position, either alone or assembled with the applicator/dispenser head 514. The cartridge 520 has an elongate nozzle 522 slightly tapered at its uppermost edge to facilitate both fitting of the cartridge cap 524 used for storage and insertion of the stopper 582 of the cartridge port 580 when fitting to the head 514. The cap 524 is of H-section for reducing the risk of damage to the nozzle 522 in the event that the cartridge 520 is dropped and has a screw thread 644 for screwing on to the complementary screw thread 642 around the base of the nozzle 522. The lower section of the cap 524, being hollow, does not interact with the one-way lock features (not shown) on the cartridge 520 and hence the cap 524 is easily removable by unscrewing.

The cartridge 520 (shown with cap removed) together with the applicator/dispenser head 514 is illustrated in perspective view in FIG. 36. The cartridge 520 has an outer ring 650, concentric with the nozzle 522, extending from the cartridge shoulder and provided with four outwardly projecting engagement ribs 532 which interact with locking clips 528 on the head 514 when the cartridge 520 is screwed on to the head.

FIG. 37 is a cross-section through the assembled cartridge and head showing the interaction between the engagement ribs 532 of the cartridge 520 and the locking clips 528 on the cartridge port 580 of the head 514 which together provide a one-way lock. Specifically, as the cartridge 520 is screwed on to the head 514 in an anti-clockwise direction the ribs 532 depress and ride over the clips 528, whereas when an attempt is made to unscrew the cartridge 520 by movement in a clock-wise direction the ribs 532 simply butt against the protruding edge of the clips 528 to prevent further unscrewing.

Many other variations are possible and while a number of examples have been chosen to illustrate the present invention, it will be apparent to those skilled in the art that various changes and modifications can be made herein without departing from the spirit and scope of the invention. Moreover, the applicator may be used to apply medicaments for any number of skin complaints affecting the scalp and other body areas, including for example dermatitis and eczema, as well as psoriasis. Combinations of features described hereinbefore, whether or not exemplified, are also regarded as falling within the scope of the invention. Thus, the foregoing description of the embodiments according to the present invention are provided for illustration only, and not for the purpose of limiting the invention.

1. A hand-held applicator for targeted self-administration of a semi-solid medicament directly to the scalp and to other body areas, the hand-held applicator comprising:
an applicator head comprising at least one elongate dispensing nozzle for contacting areas of the scalp and other body areas;
an applicator body for housing a medicament reservoir;
a pump assembly comprising at least one pump chamber, the at least one pump chamber having an inlet for receiving medicament from the medicament reservoir and an outlet for discharging medicament to the at least one elongate dispensing nozzle on the applicator head; and
an actuator that is operable on the at least one pump chamber for pumping medicament in the at least one pump chamber through the outlet and for dispensing a corresponding medicament dose from the at least one elongate dispensing nozzle onto the scalp or other body area,
wherein the applicator head further comprises an application face for spreading the dispensed medicament over a body area to be treated.

2. The hand-held applicator of claim 1,
wherein the applicator head and applicator body are fixed relative to each other in use such that movement of the at least one elongate dispensing nozzle away from the scalp or other body area during actuation may be avoided.

3-5. (canceled)

6. The hand-held applicator of claim 1, wherein the application face is disposed adjacent a dispensing nozzle.

7. The hand-held applicator of claim 1, further comprising an airless delivery system that substantially isolates medicament in the hand-held applicator from contact with air.

8. The hand-held applicator of claim 1, wherein the applicator body comprises a medicament cartridge.

9. The hand-held applicator of claim 1, further comprising a cartridge port for receiving a dispensing end of a medicament cartridge charged with medicament, the cartridge port being in fluidic communication with the inlet of the at least one pump chamber of the pump assembly.

10. The hand-held applicator of claim 9, wherein the cartridge port comprises a sealing member that seals against the neck of the cartridge and prevents leakage of medicament.

11. The hand-held applicator of claim 10, wherein the sealing member comprises a stopper insertable into the cartridge neck to displace air from the cartridge, the stopper including a central bore that permits medicament to flow from the cartridge to the at least one pump chamber via the inlet.

12. The hand-held applicator of claim 10, wherein the cartridge port further comprises a cap that engages an outer wall of the cartridge neck and fixes the sealing member with respect to the neck.

13. The hand-held applicator of claim 8, further comprising a priming insert that advances a cartridge plunger at a non-dispensing end of the cartridge along the cartridge body to displace air from the cartridge and prime the hand-held applicator with medicament prior to a first use of the held-held applicator.

14. The hand-held applicator of claim 1, wherein the applicator head, the pump assembly and the actuator together form a unitary head assembly that is connectable to a medicament cartridge, and wherein the medicament cartridge comprises the applicator body.

15. The hand-held applicator of claim 14, wherein the head assembly comprises a non-return mechanism that prevents disengagement of the head assembly from the cartridge once the cartridge has been fitted to the head assembly.

16. The hand-held applicator of claim 1, wherein each at least one elongate dispensing nozzle projects at an inclined angle with respect to the applicator body, and where more than one elongate dispensing nozzle is provided, the elongate dispensing nozzles extend parallel with each other.

17. The hand-held applicator of claim 16, wherein at least one elongate dispensing nozzle extends substantially normally from an inclined face of the applicator head.

18. The hand-held applicator of claim 16, wherein the applicator head comprises one elongate dispensing nozzle and the application face, and wherein the one elongate dispensing nozzle and the application face together comprise a substantially continuous surface of the hand-held applicator.

19. The hand-held applicator of claim 1, wherein each at least one elongate dispensing nozzle is tapered toward the nozzle tip to enhance penetration of the hair during scalp application.

20-22. (canceled)

23. The hand-held applicator of claim 1, wherein each at least one elongate dispensing nozzle comprises a nozzle stem of a substantially rigid material and a nozzle tip of a material that less rigid than the nozzle stem material.

24. The hand-held applicator of claim 23, wherein the nozzle stem of each elongate dispensing nozzle terminates in an end port and the nozzle tip of each elongate dispensing nozzle has one or more side ports in fluidic communication with the end port of the nozzle stem and from which medicament is dispensable.

25-27. (canceled)

28. The hand-held applicator of claim 1, further comprising an inlet valve that seals the inlet of the at least one pump chamber from the medicament reservoir under positive pressure in the at least one pump chamber and that opens the inlet of the at least one pump chamber under negative pressure in the pump chamber to permit medicament to flow from the medicament reservoir into the at least one pump chamber.

29. The hand-held applicator of claim 28, wherein the at least one pump chamber further comprises an outlet valve that seals the outlet from the at least one pump chamber under negative pressure in the at least one pump chamber and opens the outlet from the at least one pump chamber under positive pressure within the at least one pump chamber to permit medicament to flow from the at least one pump chamber to the at least one elongate dispensing nozzle.

30. The hand-held applicator of claim 29, wherein the inlet valve of each pump chamber, or the outlet valve of each pump chamber, or both comprises a unitary valve molding having a valve member for closing that closes the inlet or outlet, an anchor portion that secures the molding with respect to the inlet or outlet, and a spring portion between the valve member and the anchor portion that urges the valve member against the inlet or outlet to close the valve.

31. The hand-held applicator of claim 29, wherein each outlet valve comprises a resilient elastomeric material that permits outward deformation away from its respective outlet during application of a discharging force on the at least one pump chamber.

32. The hand-held applicator of claim 1, wherein each at least one pump chamber is a collapsible chamber.

33-34. (canceled)

35. The hand-held applicator of claim 1, wherein each at least one pump chamber comprises a cylinder having a longitudinal axis.

36. The hand-held applicator of claim 35, wherein the longitudinal axis of the cylinder is substantially normal to the longitudinal axis of the applicator body.

37. The hand-held applicator of claim 1, wherein the actuator comprises a plunger that displaces medicament from each at least one pump chamber and a lever arm rotatable about a fulcrum for advancing the plunger.

38-40. (canceled)

41. The hand-held applicator of claim 1, wherein the actuator comprises a rotatable plunger, and the hand-held applicator further comprises a dial that rotates the plunger whereby the plunger may be rotated to a single nozzle dispensing position in which actuation thereof results in discharge of medicament from one pump chamber and, if the applicator includes two or more pump chambers, the plunger may also be rotated to a multiple nozzle dispensing position in which actuation results in discharge from all pump chambers.

42. The hand-held applicator of claim 41, wherein the plunger is rotatable by the dial to a locking position in which actuation of the plunger results in no discharge of medicament.

43. The hand-held applicator of claim 41, wherein the rotatable plunger comprises a plunger body having a front actuating face and an actuator rod of asymmetric cross-section projecting from the rear of the plunger body and the dial has a central aperture of corresponding cross-section for receiving the actuator rod whereby rotation of the dial effects rotation of the plunger.

44. The hand-held applicator of claim 43, comprising two or more pump chambers having chamber pins extending therefrom and wherein the front actuation face of the plunger body is provided with three or more apertures, the apertures spaced in such a way that when rotated to a first plunger position the plunger face advances all chamber pins upon actuation of the actuator rod thereby to discharge medicament from all pump chambers, when rotated to a second plunger position the plunger face advances only the chamber pin associated with the pump chamber that discharges to a single nozzle, the other chamber pin being received in an aperture so that it is not advanced, and when rotated to a third plunger position the plunger face contacts none of the chamber pins, all chamber pins being received in respective apertures, so that no medicament is discharged upon actuation.

45. The hand-held applicator of claim 44, further comprising an insert interposed between the dial and the plunger body, the insert having a plurality of internal projections acting as keys that co-operate with external projections on the plunger body to permit reciprocating movement of the plunger body only when the plunger body is in pumping or locked positions.

46. The hand-held applicator of claim 1, further comprising a detachable cover that protects the application face and seals each at least one elongate dispensing nozzle when the hand-held applicator is not in use.

47. The hand-held applicator of claim 46, wherein the cover further comprises an actuator lock that prevents operation of the actuator when the cover is fitted.

48-75. (canceled)

76. A medicament cartridge for storing a medicament reservoir, the medicament cartridge having a first dispensing end adapted to fit into the cartridge port of the hand-held applicator according to claim 9.

77. The medicament cartridge of claim 76, comprising a lock structure for interlocking with a complementary structure on the hand-held applicator whereby the locking structures co-operate to permit assembly and prevent disassembly.

78. The medicament cartridge of claim 77, wherein the lock structure comprises a plurality of engagement ribs on a collar around the cartridge nozzle neck that engage with complementary locking clips on the hand-held applicator.

79. The medicament cartridge of claim 76, wherein the cartridge neck is adapted to receive and seal with a stopper of an applicator head assembly or manifold of a dispenser head to form an airtight connection after displacement of air from the cartridge neck by insertion of the stopper.

80. The medicament cartridge of claim 79, wherein the inner wall of the cartridge neck is tapered at its open end, the tapered section providing a sealing surface for a manifold sealing wall.

81. The medicament cartridge of claim 76, wherein the cartridge neck is double walled to provide a secondary chamber for receiving any medicament forced out of the medicament cartridge from between the neck and the stopper on insertion of the stopper.

82. The medicament cartridge of claim 76, comprising a tube or the like for accommodating medicament and a plunger in sealing contact with the inner tube wall, the plunger being adapted to advance up the tube wall under negative pressure created when medicament is discharged from each at least one pump chamber of the hand-held applicator.

83. The medicament cartridge of claim 82, further comprising a cartridge cap that seals the dispensing end of the medicament cartridge prior to use and wherein the cartridge cap has a dual function as a priming insert for insertion at the non-dispensing end of the medicament cartridge to advance the plunger from behind and prime the applicator for use.

84. The medicament cartridge of claim 76, further comprising an end cap at its non-dispensing end for sealing an open end of the applicator body when the medicament cartridge is for insertion into an applicator body.

85. The hand-held applicator of claim 1, further comprising a cartridge port, the hand-held applicator being fitted with a medicament cartridge having a first dispensing end adapted for fitting to the cartridge port.

86. The hand-held applicator of claim 9, further comprising a medicament cartridge having a first dispensing end adapted for fitting to a cartridge port of the hand-held applicator.

87. A method of delivering semi-solid medicament to the scalp or other body area, the method comprising:
(i) charging the medicament to an applicator body of an applicator, the applicator further comprising an applicator head fixed relative to the applicator body and having at least one elongate dispensing nozzle for contacting areas of the scalp and other body areas, a pump assembly comprising at least one pump chamber having an inlet for receiving medicament from the applicator body and one or more outlets for discharging medicament to the at least one dispensing nozzle and an actuator operable on the at least one pump chamber for pumping a dose of medicament in the pump chamber through each outlet;
(ii) priming the applicator to transfer medicament from the applicator body to the at least one pump chamber and the at least one elongate dispensing nozzle;
(iii) bringing at least one delivery nozzle chosen from the elongate dispensing nozzles into contact with the scalp or other body area after priming the applicator; and
(iv) operating the actuator to pump a dose of medicament from the pump chamber through the at least one delivery nozzle and to discharge a dose of medicament from the at least one delivery nozzle onto the scalp or other body area.

88. The method of claim 87, wherein charging the medicament comprises inserting a dispensing end of a cartridge charged with medicament into a cartridge port on the applicator, the cartridge port being in fluidic communication with the inlet to the at least one pump chamber.
